(19) United States
(12) Patent Application Publication
(10) Pub. No.: US 2025/0345150 A1
(43) Pub. Date: Nov. 13, 2025

(54) SYSTEMS AND METHODS FOR ORTHODONTIC TREATMENT PLANNING AND ORTHODONTIC APPLIANCE MANUFACTURING USING UNIVERSAL COMMANDS AND TREATMENT PROTOCOLS

(71) Applicant: Ormco Corporation, Brea, CA (US)

(72) Inventors: Evan Yifeng Tsai, Rancho Cucamonga, CA (US); Pavel Agapov, Talent, OR (US); Roman Rolinsky, Brea, CA (US); Aurthem Borodenko, Brea, CA (US); Andrey Taranov, Brea, CA (US); Vadim Bulavkin, Brea, CA (US); Ivan Karacharov, Brea, CA (US); Ekaterina Medentsova, Novi Sad (RS); Andrei Polevodov, Brea, CA (US); Aliya Gubaidullina, Brea, CA (US); Nikolay Polyakov, Brea, CA (US); Olena Yudenko, Brea, CA (US); Rafael Gaitan, Brea, CA (US); Nikita Pchelintsev, Brea, CA (US); Alexey Ignatenko, Brea, CA (US)

(21) Appl. No.: 19/199,784

(22) Filed: May 6, 2025

Related U.S. Application Data
(60) Provisional application No. 63/643,576, filed on May 7, 2024.
(60) Provisional application No. 63/739,294, filed on Dec. 27, 2024.

(51) Int. Cl. A61C 7/00; A61C 13/34; G06F 3/04815; G06F 3/0482; G06F 3/04845; G16H 20/10

(52) U.S. Cl. CPC A61C 7/002; A61C 13/34; G06F 3/04815; G06F 3/0482; G06F 3/04845; G16H 20/10

(57) ABSTRACT

A system and method for orthodontic treatment planning for a patient. The system includes a processor and memory coupled to the processor, which cause the system to display a user interface on a display. The interface provides a plurality of commands for selection. At least one command for selection is a predetermined instruction based on orthodontic nomenclature. When executed, the system receives a command into the prescription for treatment and receives a treatment protocol including a time period for application of a first command of the of the plurality of commands during a treatment period over which an orthodontic treatment plan is to be applied.

### CROSS REFERENCE TO RELATED APPLICATION

This application claims priority to U.S. Provisional App. No. 63/643,576 filed on May 7, 2024 and to U.S. Provisional App. No. 63/739,294 filed on Dec. 27, 2024, each of which is incorporated by reference herein in their entireties. This application is related to U.S. application Ser. No. 17/711,279, filed Apr. 1, 2022, the disclosure of which is incorporated by reference herein in its entirety.

### TECHNICAL FIELD

The present invention relates generally to the field of orthodontic treatment and, more particularly, to systems and methods for orthodontic treatment planning and orthodontic appliance manufacturing.

### BACKGROUND

Orthodontics is the practice of manipulating teeth to correct malocclusions between the teeth of the upper and lower dental arches. Typically, treatment of malocclusions includes the use of an orthodontic appliance that applies corrective forces to the teeth. Over time, these corrective forces coerce the teeth to move into their orthodontically correct positions.

One way of applying corrective forces to teeth is an orthodontic appliance referred to as an “aligner.” Other orthodontic appliances include orthodontic brackets that are secured to the teeth and are usable with an orthodontic archwire to apply corrective forces to a patient's teeth.

Aligners are generally supplied as a series of removable appliances that incrementally reposition the patient's teeth from their initial orientation, in which the teeth may be maloccluded, to their orthodontically correct and aesthetic orientation. Patients being treated with aligners can insert and remove the aligners at will, and therefore do not need to visit the orthodontist to advance their treatment. Rather, when the currently worn aligner has moved the teeth to at or near a final orientation for that aligner, the patient merely begins using the next aligner in the series according to a treatment plan. In that regard, each aligner in the series differs from all other aligners in the series.

To fabricate aligners or braces for a particular patient, the orthodontist first constructs a computer model of the patient's dentition. This model may be generated, for example, by taking an impression of the patient's dentition and then scanning the impression to digitize the impression for manipulation in a computer. Alternatively, the clinician may directly scan the patient's teeth with an intraoral scanner. The scanned data is then used to construct the computer model including model teeth. In each case, the computer model includes one or more model teeth (preferably all the patient's teeth) in a model of the patient's upper and/or lower jaws.

Once the computer model has been constructed, the orthodontist may manipulate individual ones of the model teeth to ultimately determine a target orientation of each tooth that provides a corrected dentition for each respective jaw. The target orientation ideally addresses any malocclusion and provides an aesthetic smile. Multiple computer models may be generated prior to treatment. Each model may include a unique orientation of one or more model tooth in the model dentition and may successively and incrementally reposition one or more model teeth from an initial orientation to a target orientation according to a treatment plan.

The incremental repositioning of the model teeth is then reproduced in a series of fabricated molds of the teeth. An aligner is formed from each fabricated mold. Where there are multiple molds, a set of aligners is manufactured with each aligner being unique to one of the molds. When worn by a patient, each aligner imposes forces on the patient's teeth during orthodontic treatment. The patient's teeth may be moved incrementally from initial to target positions and orientations according to the treatment plan as determined by the computer models. In this way, treatment moves the patient's teeth in a series of stages from an initial orientation that generally corresponds to the initial orientation of the computer model to a final orientation that ideally corresponds to the target orientation of the computer model.

Orthodontists often directly or indirectly prepare each stage of the treatment plan by providing specific instructions to the orthodontic appliance manufacturer. These instructions may include treatment goals for a patient. Those goals are a result of the orthodontist's examination of the patient's condition and are based on the orthodontist's experience and preferred treatment methods. Treatment goals may include specific instructions for individual tooth movement and may include a specific order of tooth movement by which the goals are to be obtained. The specific instructions are in the form of a text-based description prepared by the orthodontist and transmitted to the appliance manufacturer. The instructions are then interpreted by a technician at the appliance manufacturer. The technician is responsible for preparing the digital treatment plan based on the text-based description. Once prepared, the treatment plan may be transmitted to the orthodontist for final approval prior to manufacturing any orthodontic appliances. Once approved, the corresponding appliances designed to treat the patient's malocclusion are manufactured and shipped to the orthodontist or patient for use by the patient.

While successful, there are significant drawbacks to current treatment planning. Thus, improved systems, and methods are needed in orthodontic treatment planning of orthodontic appliances.

### SUMMARY

The present invention overcomes the shortcomings and drawbacks of methods and systems for treatment planning heretofore known for use in orthodontic treatment. While the invention will be described in connection with certain embodiments, it will be understood that the invention is not limited to those embodiments. On the contrary, the invention includes all alternatives, modifications and equivalents as may be included within the spirit and scope of the present invention. In one aspect of the invention, there is a system for orthodontic treatment planning for a patient. The system includes a processor and memory coupled to the processor. The memory is configured to store computer-program instructions that, when executed by the processor, cause the system to display a user interface on a display. The user interface is for a user to input a prescription for treatment of the patient. In that regard, the user interface displays a plurality of commands for selection. At least one command of the plurality of commands for selection is a predetermined instruction based on orthodontic nomenclature for moving or modifying one or more of a patient's teeth. When executed by the processor, the computer-program instructions cause the system to receive a first command of the plurality of commands into the prescription for treatment. Receipt of the first command into the prescription for treatment defines a treatment protocol for application of the prescription for treatment to the patient's teeth. The treatment protocol includes a first time period during which the first command is applied to the patient's teeth, the first time period being during a treatment period over which the prescription for treatment is to be applied to the patient's teeth.

In one embodiment, the memory is configured to store computer-program instructions that, when executed by the processor, cause the system to, after receipt of the first command, receive a second command of the at least one command of the plurality of commands into the prescription for treatment. Receipt of the second command into the prescription of treatment adds a second time period to the treatment protocol. The second time period determines when the second command is applied to the patient's teeth. The first time period is before or is simultaneous with the second time period.

In one embodiment, the treatment period of the treatment protocol is defined by a plurality of steps. Receipt of the first command into the prescription for treatment sets the first time period to at least one step of the plurality of steps.

In one embodiment, the treatment protocol is defined by a plurality of steps. Receipt of the first command into the prescription for treatment sets the first time period to be equal to all steps of the plurality of steps.

In one embodiment, the treatment protocol is defined by a plurality of steps including a first step and a second step. Receipt of the first command sets the first time period as the first step and the second step. Receipt of the second command sets the second time period as the first step whereby the first command and the second command are applied to the patient's teeth simultaneously during the first step.

In one embodiment, the second time period does not include the second step whereby the first command is applied to the patient's teeth in the second step and the second command is not applied to the patient's teeth in the second step.

In one embodiment, when executed by the processor, the computer-program instructions cause the system to generate a plurality of stages so that one or both the first time period and the second time period are defined by one or more of the plurality of stages.

In one embodiment, when executed by the processor, the computer-program instructions cause the system to display, in the user interface, an integrated staging chart. The integrated staging chart depicts at least the first command and the first time period as one or more of the plurality of stages. The integrated staging chart defines at least a portion of the orthodontic treatment plan.

In one embodiment, each stage corresponds to planned orthodontic treatment with an appliance. For example, the appliance is an aligner.

In one embodiment, the integrated staging chart displays the first time period and the second time period sequentially with the first command being shown as being applied to the patient's teeth before the second command.

In one embodiment, when executed by the processor, the computer-program instructions cause the system to display, in the user interface, the plurality of steps overlaid with the plurality of stages.

In one embodiment, when executed by the processor, the computer-program instructions cause the system to display, on the user interface, a message that the displayed command of the plurality of commands is locked against being received in the prescription for treatment.

In one embodiment, when executed by the processor, the computer-program instructions cause the system to display, on the user interface, a virtual model of teeth and a video demonstration of at least one of the first command and the second command as applied to the virtual model of teeth.

In one embodiment, the first time period is displayed horizontally on the display or the first time period is displayed vertically on the display.

In one embodiment, when executed by the processor, the computer-program instructions cause the system to display, on the user interface, a virtual model of teeth and controls operable by the user to cause the processor to simulate application of tooth movement according to at least one of the first command and the second command to the virtual model of teeth.

In one embodiment, when executed by the processor, the computer-program instructions cause the system to display, in the user interface, a virtual model of patient's teeth and one or more controls operable by the user to cause the processor to simulate application of the prescription for orthodontic treatment of the patient to the virtual model of the patient's teeth.

In one embodiment, wherein the first command includes one or more tooth movement parameters, when executed by the processor, the computer-program instructions cause the system to display, in the user interface, one or more controls linked to one or more teeth in the virtual model. The controls are operable by the user to cause the processor to modify tooth movement parameters in the first command and/or in the second command.

In one embodiment, when executed by the processor, the computer-program instructions cause the system to display, on the user interface, an integrated staging chart. The integrated staging chart depicts the first command and the second command and the treatment protocol. The integrated staging chart defines at least a portion of the orthodontic treatment plan.

In one embodiment, when executed by the processor, the computer-program instructions cause the system to display, in the user interface, the at least one command of the plurality of commands in a respective rectangular-shaped border encircling only the predetermined instruction.

In one embodiment, each rectangular-shaped border of the respective rectangular-shaped borders encircling the predetermined instruction overlap to form two or more layers of commands.

In one embodiment, each rectangular-shaped border of the respective rectangular-shaped borders encircling the predetermined instruction overlap to form two or more layers of commands.

In one embodiment, when executed by the processor, the computer-program instructions cause the system to display, in the user interface, a layer selection bar operable by the user to select any single one or multiples of the two or more layers of commands for display.

In one embodiment, the system further includes appliance manufacturing equipment configured to manufacture an appliance based on the prescription for treatment and the treatment protocol. When executed by the processor, the computer-program instructions cause the system to transmit the prescription for treatment and the treatment protocol to the appliance manufacturing equipment and the appliance manufacturing equipment reads the prescription for treatment.

In one embodiment, when executed by the processor, the computer-program instructions cause the system to display, in the user interface, at least one keyframe at an end of at least one step of the plurality of steps.

In one embodiment, when executed by the processor, the computer-program instructions cause the system to convert the prescription for treatment and the treatment protocol into machine-readable code for use by a processor of a computer.

In one embodiment, when executed by the processor, the computer-program instructions cause the system to save the prescription for treatment and the treatment protocol in the machine-readable format.

In one aspect of the invention there is a computer-implemented method of creating an orthodontic treatment plan applicable to teeth of a patient. The method includes receiving a digital model of a patient's teeth in a first arrangement. The method further includes displaying a plurality of commands from a library of commands. The library of commands is predetermined and at least one command of the displayed plurality of commands is based on orthodontic nomenclature for moving or modifying one or more of the patient's teeth. The method further includes placing a first command of the at least one command into a prescription for treatment. Placing the first command defines a treatment protocol for application of the prescription for treatment to the patient's teeth. The treatment protocol includes a first time period that determines when the first command is applied to the patient's teeth during a treatment period over which the prescription for treatment is to be applied to the patient's teeth.

In one embodiment, after placing the first command, the method further includes placing a second command of the at least one command of the plurality of commands into the prescription for treatment. Placing the second command into the prescription of treatment adds a second time period to the treatment protocol. The second time period determines when the second command is applied to the patient's teeth. The first time period is before or is simultaneous with the second time period.

In one embodiment, the treatment protocol is defined by a plurality of steps, and placing the first command into the prescription for treatment sets the first time period to at least one step of the plurality of steps.

In one embodiment, the treatment protocol is defined by a plurality of steps, and placing the first command into the prescription for treatment sets the first time period to all steps of the plurality of steps.

In one embodiment, the plurality of steps includes a first step and a second step. Placing the first command sets the first time period to be equal to the first step and the second step. Placing the second command sets the second time period to be equal to the first step whereby the first command and the second command are applied to the patient's teeth simultaneously during the first step.

In one embodiment, placing the second command sets the second time period to be equal to the first step only whereby the first command is applied to the patient's teeth in the second step and the second command is not applied to the patient's teeth in the second step.

In one embodiment, the method further includes generating a plurality of stages so that one or both the first time period and the second time period are defined by one or more of the plurality of stages.

In one embodiment, the method further includes displaying an integrated staging chart depicting the first command and the second command and the plurality of stages. The integrated staging chart defines at least a portion of the orthodontic treatment plan.

In one embodiment, each stage of the plurality of stages corresponds to planned orthodontic treatment with an appliance.

In one embodiment, displaying the integrated staging chart includes displaying the first time period and the second time period sequentially with the first command being shown as being applied to the patient's teeth before the second command.

In one embodiment, the method further includes displaying the plurality of steps overlaid with the plurality of stages.

In one embodiment, placing one of the at least one commands into the prescription for treatment is prevented.

In one embodiment, the method further includes displaying the treatment protocol with increasing time shown horizontally on the display or displaying the treatment protocol with increasing time shown vertically on the display.

In one embodiment, the method further includes displaying a virtual model of teeth and controls operable by the user to operate the virtual model. When activated by the controls, tooth movement according to at least one of the first command and the second command to the virtual model of teeth is simulated.

In one embodiment, the method further includes displaying a virtual model of patient's teeth, and simulating application of the prescription for orthodontic treatment of the patient to the virtual model of the patient's teeth.

In one embodiment, the first command and/or the second command include one or more tooth movement parameters. The method further includes displaying one or more controls selectable by the user and configured to move one or more teeth in the virtual model of the patient's teeth. The method further includes selecting the one or more controls and moving the one or more teeth with the one or more controls which modifies one or more tooth movement parameters in the first command and/or in the second command.

In one embodiment, the method further includes displaying the at least one command of the plurality of commands in a respective rectangular-shaped border encircling the predetermined instruction.

In one embodiment, each of the respective rectangular-shaped borders encircling the predetermined instruction of the at least one command overlaps to form two or more layers of commands. The method further includes displaying the two or more layers of commands.

In one embodiment, the method further includes displaying a layer selection bar operable by a user to select any single one or multiples of the two or more layers of commands for display.

In one embodiment, the method further includes converting the prescription for treatment and treatment protocol into a machine-readable code for use by a processor of a computer.

In one embodiment, the method further includes saving the prescription for treatment and the treatment protocol in the machine-readable format.

### BRIEF DESCRIPTION OF THE DRAWINGS

The accompanying drawings, which are incorporated in and constitute a part of this specification, illustrate embodiments of the invention and together with the detailed description given below, serve to explain various aspects of the invention.

FIG. 1 is a schematic diagram illustrating one embodiment of an orthodontic treatment planning and manufacturing system.

FIG. 1A is a schematic view of the system of FIG. 1 and according to one embodiment of the invention.

FIG. 2 is an exemplary flow according to one embodiment of the system.

FIG. 3 is an exemplary user interface for use with a system of FIG. 1.

FIGS. 4A, 4B, 5, 6, 7, 8, 9, 10, 11, and 12 are exemplary user interfaces for use with a system of FIG. 1.

FIGS. 13, 14, and 15 are exemplary user interfaces for use with a system of FIG. 1.

FIGS. 16, 17, 18, 19, 20, 21, 22, 23, 24, 25, 26, 27, and 28 are exemplary integrated staging interfaces for use with a system of FIG. 1.

FIGS. 29, 30, and 31 are exemplary displays for use with a system of FIG. 1.

FIG. 32 is an exemplary user interface for use with a system of FIG. 1.

FIG. 33 is an exemplary integrated staging chart according to embodiments of the invention.

FIGS. 34, 35, 36, 37, 38, 39, 40, and 41 are exemplary user interfaces in accordance with embodiments of the invention.

FIGS. 42 and 43 are orthodontic appliances in the form of an aligner and an orthodontic bracket, respectively, according to embodiments of the invention.

### DETAILED DESCRIPTION

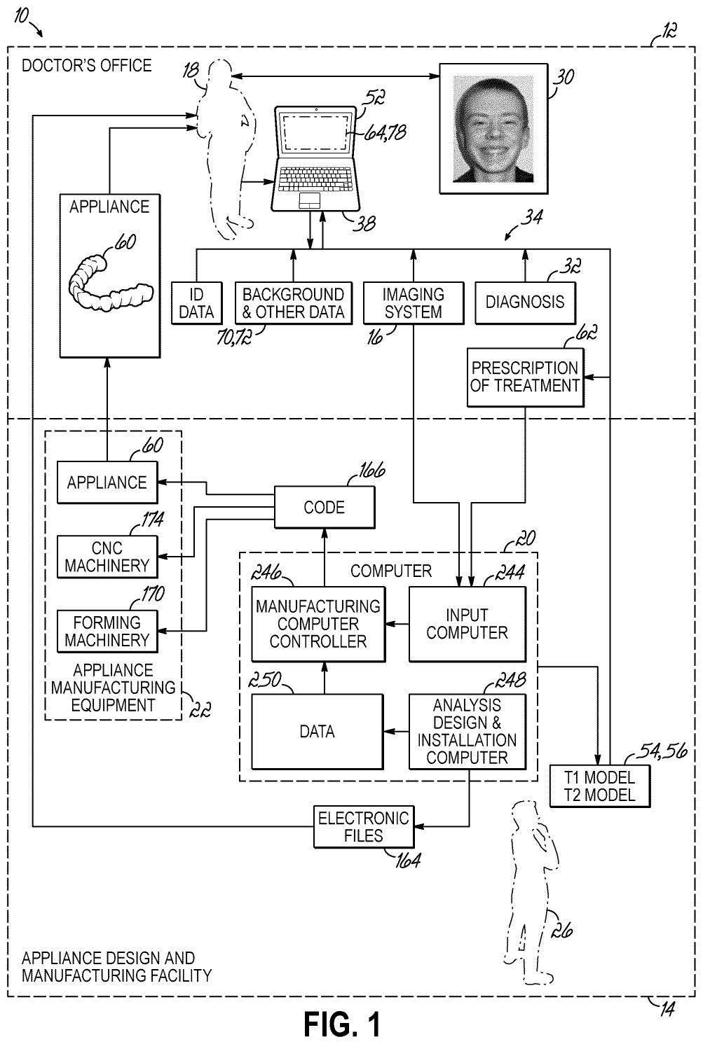

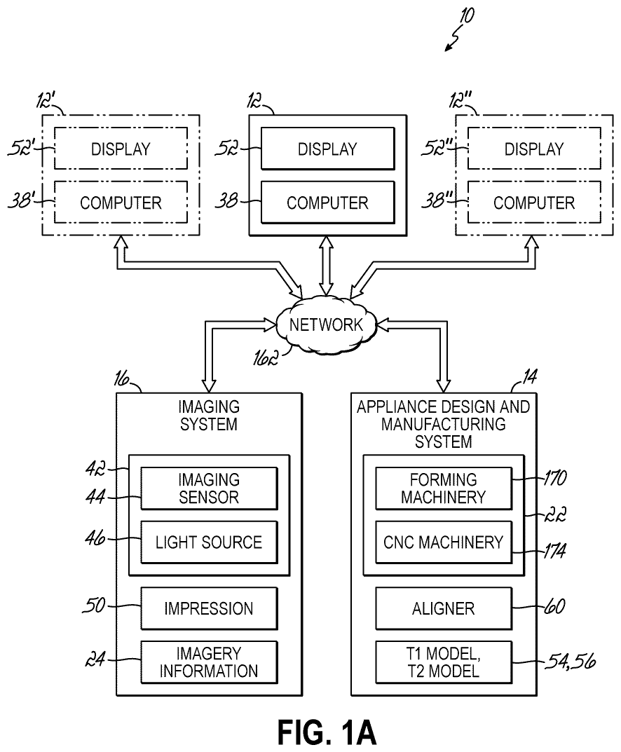

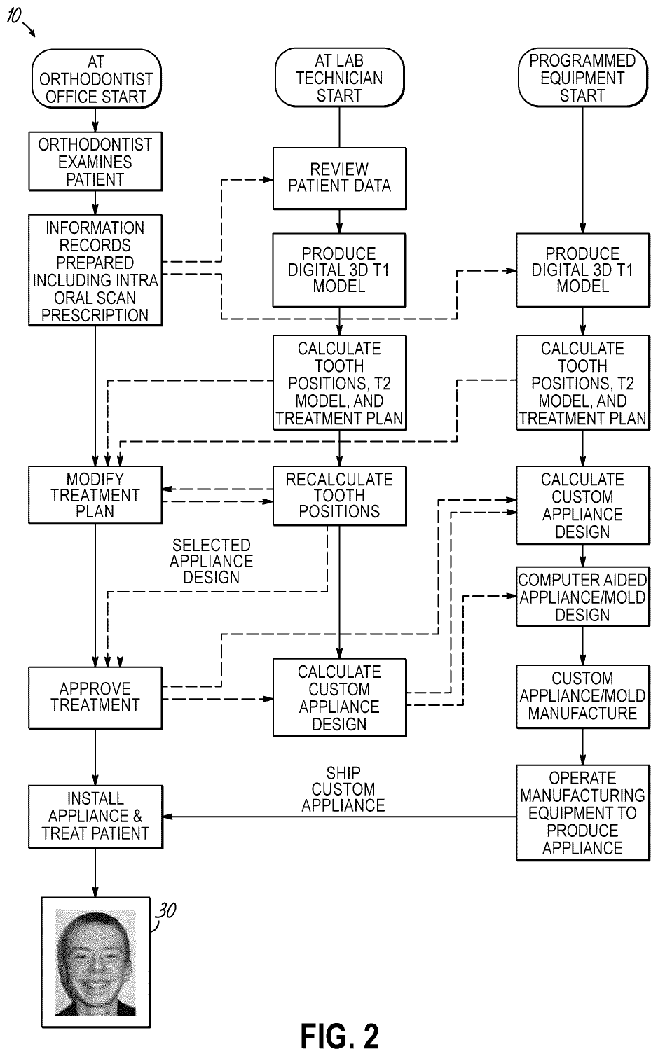

An exemplary embodiment of an orthodontic treatment planning and manufacturing system 10 is shown in FIGS. 1, 1A, and 2. Embodiments of the orthodontic treatment planning and manufacturing system 10 address problems identified with current orthodontic treatment planning and manufacturing. One exemplary problem is disclosed and addressed in commonly-owned U.S. application Ser. No. 17/711,279 (“the '279 application”), which is incorporated by reference herein in its entirety. As a summary of that problem, the orthodontist may prepare specific written instructions for patient treatment. The written instructions for a patient's orthodontic treatment is referred to as a prescription. The prescription is intended to provide individualized treatment goals by desirable tooth movement. Ultimately, a treatment plan for orthodontic treatment of the patient with orthodontic appliances is constructed from the prescription. While the prescription is specified by an orthodontist, the treatment plan, according to which the patient's teeth are moved, may be prepared by a designer and/or an appliance manufacturer. In a process of preparation of the treatment plan, a technician at the appliance manufacturer reviews the orthodontist's written instructions. Although embodiments of the invention are not limited to any relationship between the technician, who reviews the written instructions, and an appliance manufacturer, the technician may be employed by the appliance manufacturer. The technician may be responsible for incorporating the orthodontist's written instructions into an initial treatment plan for treating the patient with appliances. The technician therefore interprets the orthodontist's written instructions. Based on that interpretation, the technician then develops the initial treatment plan. It was discovered that the technician's interpretation of the orthodontist's written instructions frequently introduces unintentional deviations between the orthodontist's intended treatment and an initial treatment plan. To correct the unintended deviations, the initial treatment plan must be reworked. Reworking prolongs the period between diagnosis and beginning of orthodontic treatment and is therefore not efficient.

In accordance with embodiments of the present application, inventors identify and provide solutions to another problem with current treatment plan development methodology. Generally, current methods lack processes or guidance on how a prescription is to be executed with respect to a patient. The technician is therefore left to apply the prescription to the patient based on training, based on prior experience with the specific orthodontist, and based on intuition. Stated another way, a prescription often only generally states or infers a specific sequence of tooth movement while the resulting treatment plan requires specific tooth movement and relative timing of that movement. There is therefore a lack of information between the general tooth movement, which is often in the form of treatment goals, specified in the prescription and the need for specific tooth movement information in a treatment plan that is actually applied to the patient's malocclusion. Lack of this information makes application of the prescription to the patient difficult and in some cases impossible.

As an example, a technician may properly interpret an orthodontist's prescription, but the technician may lack information or guidance in the prescription necessary to construct a treatment plan from that prescription. This often results in the technician interpolating and/or extrapolating to create a treatment protocol by which the prescription is applied to the patient. This methodology in which the technician is left to interpolate and/or extrapolate the intent of the orthodontist as set forth in the prescription often results in back-and-forth communications between the technician and the orthodontist on how the prescription is to be applied to a specific patient. This back-and-forth communication, while productive, is a source of inefficiency and causes a delay between an initial patient consultation and treatment. Embodiments of the invention are intended to fill an information gap between the prescription and how the prescription is to be applied during orthodontic treatment planning. Embodiments of the system 10 therefore create planning and treatment efficiencies not before achieved in orthodontic treatment planning.

Embodiments of the invention, such as the orthodontic treatment planning and manufacturing system 10 shown in FIGS. 1-3, solve those problems and others. Misinterpretation of the orthodontist's instructions are eliminated by utilizing universal commands described in the '279 application. Through those universal commands, an orthodontist communicates with a technician and/or directly with a prescription system, which may include communication directly with a machine. Communication is via a standardized language having terms that are rooted in orthodontic treatment. The universal commands may be the only communication of a patient's prescription between the orthodontist and the technician and/or manufacturer. However, additional written notes may accompany the commands. The universal commands are one, two, or three-word instructions for orthodontic treatment with which the orthodontist may build a treatment prescription. The commands may be in verb or noun form. Each universal command has or is given a specific meaning in orthodontic treatment. That meaning is understandable by the orthodontist and by a technician and/or by a manufacturing facility/system to which the commands represent software code. In essence, universal commands provide a standardized, common language between an orthodontist and a technician and/or between an orthodontist and a manufacturing facility/system, much like commands in software programming. With respect to automatic reading of the prescription by a manufacturing system, a technician's participation in the development of a treatment plan may be minimized or eliminated. In any case, the commands eliminate a technician's subjective interpretation of the prescription.

Embodiments of the invention may additionally define a treatment protocol for application of the universal commands. Advantageously, with the protocol, an order of execution of single ones of the universal commands or groups of universal commands is established. Thus, embodiments of the invention provide tools and information for constructing how the prescription is to be applied to a particular patient. As an example, a prescription may establish a protocol of one or more steps for application of the universal commands to the patient's teeth. The steps provide a general order as to how the commands are to be applied to the patient. The technician may then construct a staging sequence from the steps, for example, for application of orthodontic aligners to treat the patient. Other advantages and benefits of the orthodontic treatment planning and manufacturing system 10 according to embodiments of the invention include, for example, automatic manufacturing of appliances via a computerized manufacturing system. Further, embodiments of the present invention may provide visual analysis of a treatment protocol or portions of the treatment protocol in relation to orthodontic treatment from at or near its beginning (e.g., T1) to at or near its end (e.g., T2).

With reference to FIG. 1, in an exemplary embodiment and in general, the orthodontic treatment planning and manufacturing system 10 includes a plurality of universal commands for communication of treatment information between an orthodontist's office 12 and an orthodontic appliance designing and manufacturing system 14 and so is similar to the system disclosed and claimed in the '279 application in that respect. With reference to FIGS. 1 and 1A, the orthodontic treatment planning and manufacturing system 10 may be distributed among a plurality of locations, such as between the office 12 and manufacturing system 14 and multiple other offices 12′ and 12″ (FIG. 1A). The orthodontic treatment planning and manufacturing system 10 may additionally include an imaging system 16, shown for example in FIG. 1, at the orthodontist's office 12. Further, as is schematically shown in FIG. 1A, the imaging system 16 may be at a location separate from orthodontists' offices 12, 12′, and 12″. Embodiments of the invention are not, however, limited to any number of locations. For example, the orthodontic treatment planning and manufacturing system 10 may incorporate each of the office 12, manufacturing system 14, and the imaging system 16 at a single location.

With reference to FIG. 1, communication and decision making in the operation of the orthodontic treatment planning and manufacturing system 10 may be distributed between people and/or locations, including (1) an orthodontist 18 located at the office 12, (2) data gathering and processing equipment 20 located at the system 14 (as shown) and/or at the office 12, and (3) the lab operator or technician 26 located at the system 14 in which appliance manufacturing equipment 22 is housed. Any of the orthodontist 18, the technician 26, or another person may use one or more portions of the system 10 disclosed herein. These three paths are represented by the three columns in FIG. 2. The decisions of the orthodontist 18 are illustrated at the left column, decisions and steps performed by a technician 26 or by equipment 20 at the manufacturing system 14 are illustrated in the center column, and the processes of the manufacturing equipment are illustrated at the right column. The dashed lines in FIG. 2 represent interactions, including exchanges of universal commands and/or treatment protocol information. The orthodontist 18 and the technician 26 may be generally referred to as a “clinician” or “user” herein unless otherwise specifically stated and is not limiting to embodiments of the invention.

In one embodiment of preparing a treatment plan for a patient, the orthodontist 18 examines a patient 30 at the office 12 and makes a diagnosis 32 for orthodontic treatment. That diagnosis 32 may be reduced in form to one of several data records 34 and entered in computer 38. The records 34 are generated as part of the case information necessary to determine the patient's condition, prescribe the appropriate treatment, and specify the orthodontic appliances to implement the prescribed treatment. The records 34 may include information identifying the patient 30, anatomical data from the patient 30, and other background information.

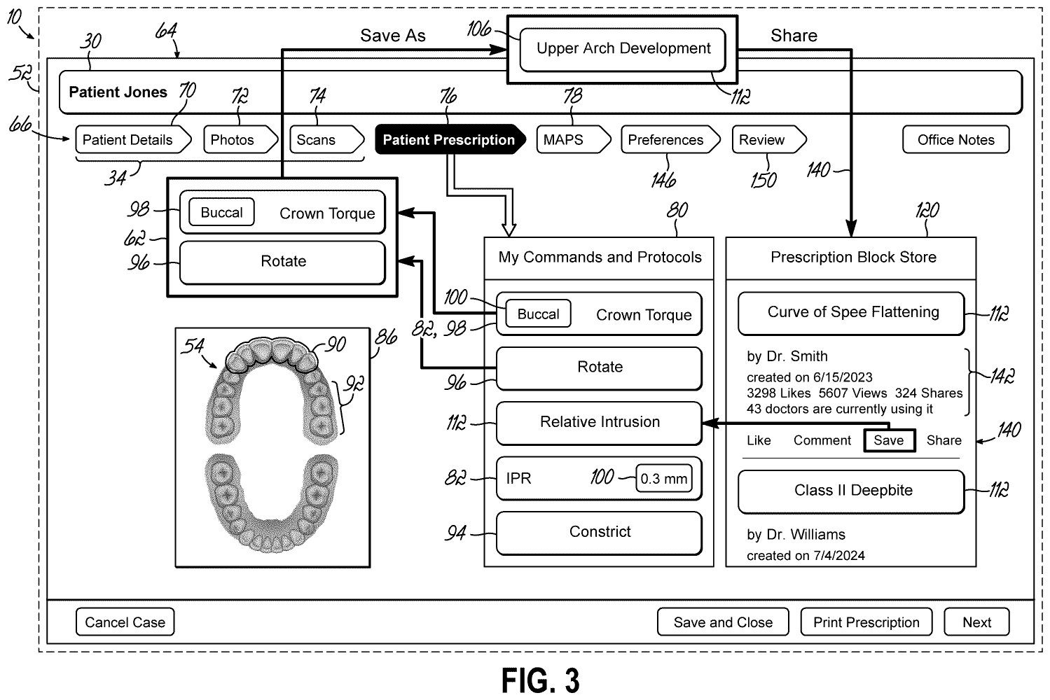

Referring to FIGS. 1 and 3, examination of the patient 30 by the orthodontist 18 involves the traditional application of the skill, knowledge, and expertise of the orthodontist 18, and results in the creation of a detailed anatomical record of the shape and initial maloccluded locations of the teeth in the patient's mouth as well as the jaw structure of the patient 30. This detailed anatomical record may include imagery information 24 of the patient's jaws from imaging system 16. The orthodontist 18 or someone under their direction may utilize the imaging system 16 to gather the imagery information 24 (FIG. 1A) from the patient 30. For example, a clinician may insert at least a portion of a wand 42 into the patient's mouth. Using a light source 44 and an imaging sensor 46, the clinician may capture data of all or selected ones of crowns of the patient's teeth. Data may include surface imagery and/or volumetric imagery (i.e., volumetric data acquired from, for example, cone beam computed tomography (CBCT) or similar device not shown) of one or more of the patient's teeth. Alternatively, surface imagery may be captured from an impression 50 of the patient's teeth. The imagery information 24 may be viewable on a display 52 coupled to computer 38 and may be transferred to the manufacturing system 14 at which virtual 3D models, referred to as a T1 model 54 and a T2 model 56 may be prepared by the technician 26.

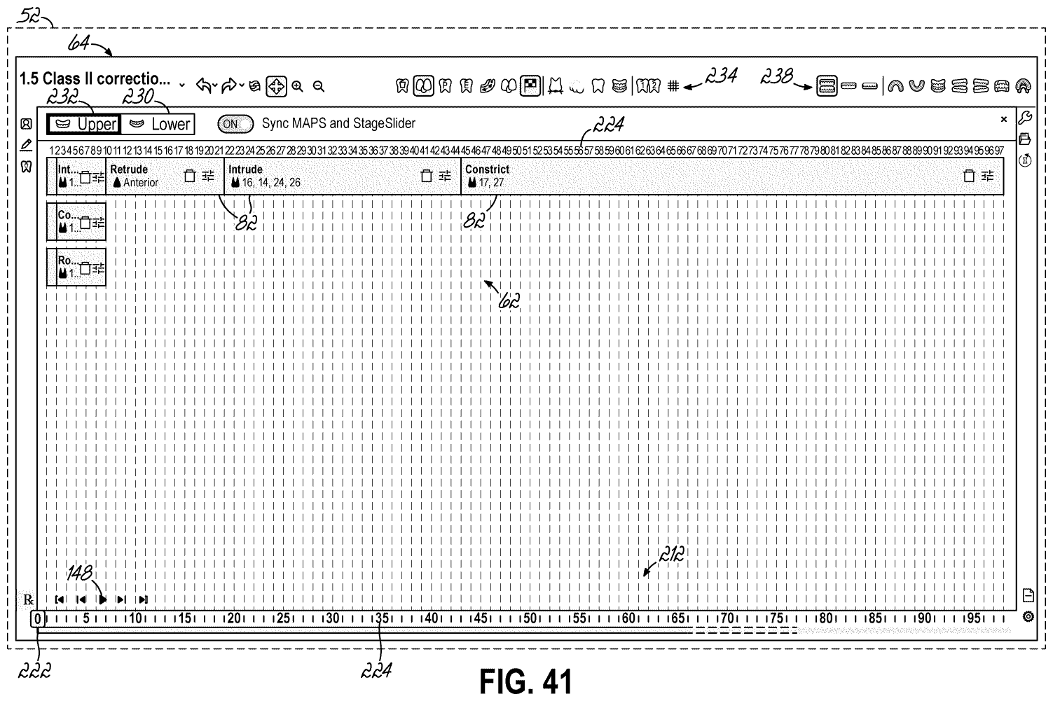

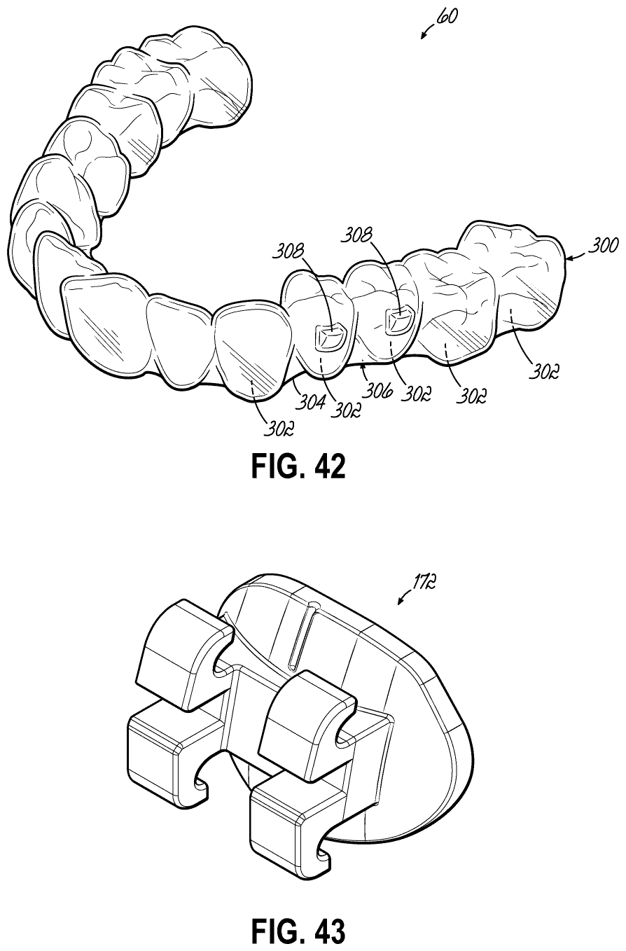

The orthodontist 18 determines the general type of orthodontic appliance (e.g., orthodontic aligner 60 (FIG. 41) and/or orthodontic bracket 172 (FIG. 42)) with which the patient 30 is to be treated. As shown, in the orthodontic appliance treatment and manufacturing system 10, the exemplary appliance is an aligner 60 (see, e.g., FIG. 1). To initiate production of the aligner 60 (typically as one of a series of aligners), the doctor's office 12 transmits the imagery information 24 from the imaging system 16 to the manufacturing system 14 along with the other data records 34, including a prescription 62 in which the orthodontist 18 sets forth one or more universal commands for inclusion in an initial treatment plan for the patient 30.

With reference to FIGS. 1 and 3, the prescription 62 is generated via a user interface 64 on display 52. The orthodontist 18 enters the prescription 62 via interface 64 into computer 38 for transmission from the computer 38 to the manufacturing system 14. The user interface 64 is designed to prompt the orthodontist 18 for information necessary to treat a particular patient, particularly building the prescription 62. As shown in FIG. 3, in one embodiment, the user interface 64 includes a flowchart 66 by which the user interface 64 guides the orthodontist 18 in preparing data records 34 required to treat the patient 30. For example, the flowchart 66 guides preparation of information concerning each of “Patient Details” 70, “Photos” 72, “Scans” 74 (e.g., imagery information 24), “Patient Prescription” 76, “MAPS” 78, “Preferences” 146, and “Review” 150. User interfaces according to embodiments of the invention are not limited to the user interface 64 shown in FIG. 3.

With continued reference to FIG. 3, as an example, according to flowchart 66 the orthodontist or technician enters each of the patient details 70, patient photos 72 and intraoral scans 74. At Patient Prescription 76, the orthodontist 18 prepares the patient's prescription 62 to be used for developing an initial orthodontic treatment plan. Details of building the patient's prescription 62 during the Patient Prescription 76 stage are found in the '279 application. A summary of building the prescription 62 is described in this application to aid in the description of embodiments of the orthodontic appliance treatment and manufacturing system 10, including MAPS 78, disclosed herein. While the orthodontic appliance treatment and manufacturing system 10 described in this application may be utilized collectively with the system disclosed in the '279 application in a single system, embodiments of the orthodontic appliance treatment and manufacturing system 10 disclosed herein are not limited to any single one or combination of details of the system and building the patient's prescription 62 disclosed in the '279 application.

In one embodiment, to facilitate preparation, the user interface 64 depicts one or more selectable universal commands 82 or user-defined commands or protocols 112 (commands and protocols may be referred to generally herein as commands) by which the orthodontist may build the prescription 62 for orthodontic treatment of the patient 30 (e.g., “Patient Jones”). Universal commands 82 are predetermined instructions, such as a one-word, two-word, or three-word instruction, typically in the form of, or including, a verb or a noun. While one-word, two-word, and three-word instructions are disclosed, embodiments of the invention are not limited thereto, unless otherwise specified. The words for each universal command are selected from orthodontic treatment nomenclature. The assigned meaning and thus function of each command 82 is based on an industry definition. However, embodiments of the invention are not limited to orthodontic treatment nomenclature. By way of further example, the commands 82 may include terms and/or symbols unrelated to orthodontic treatment, if the meaning of those terms and symbols is communicated with the orthodontist 18 and the technician 26. Protocols 112 may be combinations of universal commands 82 that the orthodontist builds initially for their own use and then saves in the orthodontic treatment planning and manufacturing system 10. Because protocols 112 themselves are constructed of universal commands 82, protocols 112 may be referred to herein as commands herein. Protocols 112 may be used alone or in combination with commands 82 and/or in combination with other protocols 112. In any respect, in the orthodontic treatment planning and manufacturing system 10, the meaning of each command 82, 112 is known by each of the orthodontist 18 and the technician 26.

With reference to FIG. 3, the commands 82, 112 are individually selectable, such as from a My Commands Protocol block 80 or from a store 120 for building the prescription 62. The orthodontist may select one or more of the commands 82, 112 by dragging and dropping the command 82, 112 from the protocol block 80 or the store 120 of commands to the prescription 62. In the exemplary embodiment, the user interface 64 provides an image area 86 which may depict a patient image. By way of example only, and not limitation, the T1 model 54 for the patient may be shown in the image area 86. In the interface 64, the commands 82, 112 displayed in the protocol block 80 may be linked to a specific tooth 90, a segment of teeth 92 in the T1 model 54, or the entirety of the T1 model 54 (e.g., the entire dentition, including both arches) shown in the image area 86. For example, if the orthodontist selects the upper anterior teeth 90 on the T1 model 54 in the image area 86, the commands 82, 112 available for selection in the protocol block 80 and store 120 may differ from the commands 82, 112 available for selection in the protocol block 80 and store 120 if the orthodontist 18 selects one or more of the teeth 92 for treatment. In this way, the commands 82, 112 available for selection may be linked to and/or varied by the image displayed in the image area 86. Advantageously, this may make building the prescription 62 easier as only commands 82, 112 relevant to the image area 86, e.g., the selected teeth 90 or 92, are displayed in the protocol block 80 and/or the store 120.

Continuing that example, in FIG. 3, the orthodontist 18 may select an anterior segment (canine to canine) 90 of an upper arch in the T1 model 54. With that selection, the My Commands and Protocols block 80 may list relevant commands 82, 112 for the anterior segment 90. Commands 82 and 112 relevant to the anterior segment 90 may also be shown in the store 120, by which the orthodontist 18 may access additional, new commands 82, 112. The orthodontist 18 may select the “Constrict” command 94, “Rotate” command 96, “Crown Torque” command 98 for inclusion in the prescription 62. Each of these commands 94, 96, 98 is selected, such as dragging and dropping, from the prescription block 80 to the prescription 62. The Constrict command 94 means moving teeth towards a palatal side of an arch form, such as the upper arch in the T1 model 54. The Rotate command 96 means rotating a tooth mesiodistally around its long axis. The Crown Torque command 98 means changing the torque of the tooth (a third order movement around an x-axis) while keeping the root apex stationary. This occurs when a force is applied and causes movement of the crown and the root of a tooth in a bucco-lingual direction. Constrict command 94 and Rotate command 96 are examples of a one-word instruction. Crown Torque command 98 is an example of a two-word instruction. When Crown Torque command 98 is selected, the orthodontic treatment planning and manufacturing system 10 via the interface 64 may prompt the orthodontist, or the orthodontist may optionally select, a command parameter 100, such as “Buccal,” as shown, or “Lingual”, in which the selected crown is to be torqued, e.g., buccally or lingually, respectively.

With reference to FIG. 3, in one embodiment, many of the exemplary commands 82 include one or more of the command parameters 100, such as a direction “Buccal” or “Lingual” for Crown Torque command 98. By way of example only and not limitation, one or more of the commands 82 may include one command parameter 100 and other commands 82 may include two command parameter 100. Further, in one embodiment, some commands lack command parameters. The orthodontic treatment planning and manufacturing system 10 may prompt the orthodontist 18 to fill in or select the command parameter 100 simply by clicking on it and then selecting a variable from a list or entering the desired distance.

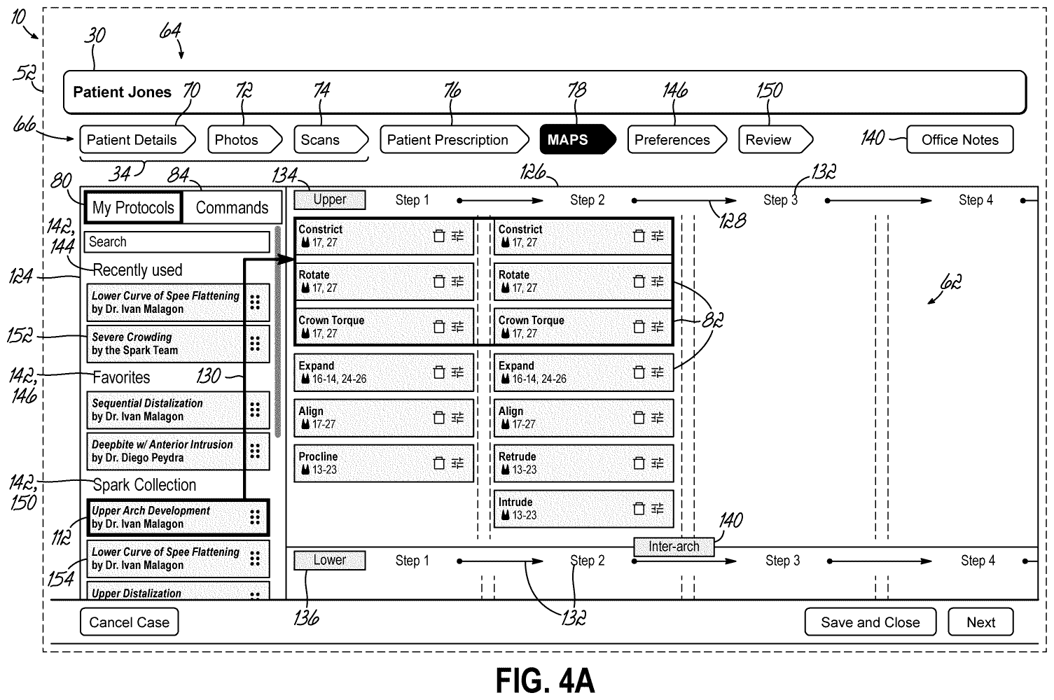

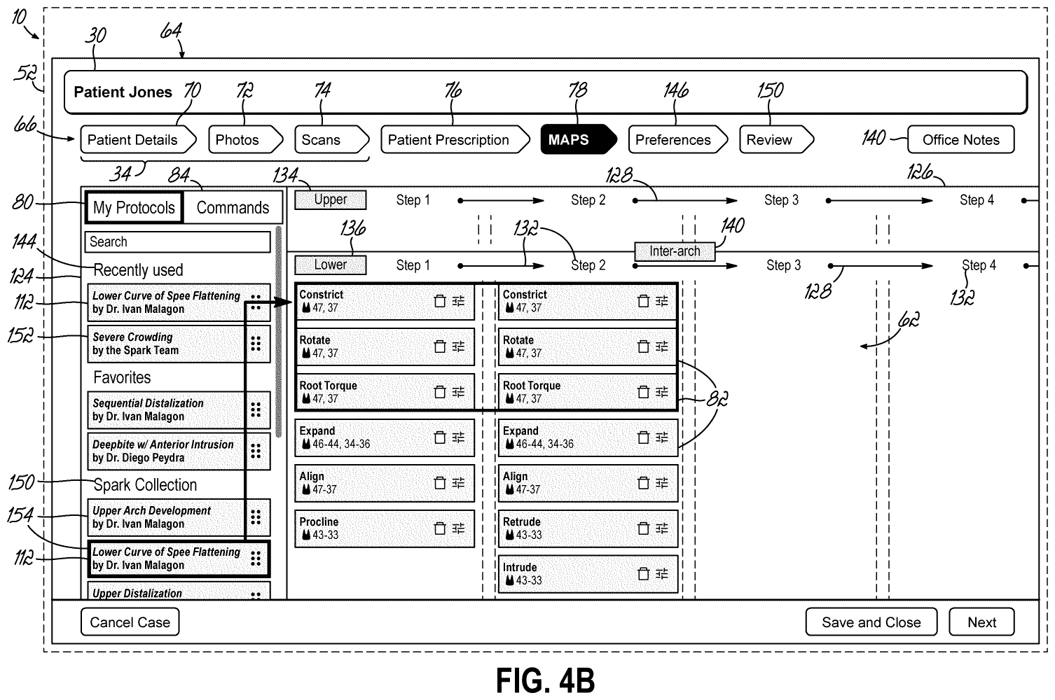

As another example and with reference to FIG. 3, the IPR command 82 means interproximal reduction, which refers to a procedure for modifying, for example, removing, a portion of the tooth surface. The command parameter 100 is a dimension and is shown as “0.3 mm” and is the thickness of interproximal reduction required on the selected tooth. The orthodontist 18 may additionally or optionally set timing for when the IPR is to occur, timing is generally described below. The value of any parameter for IPR may default to “Delay as much as possible” or other timing may be set by the orthodontist 18, for example, to “at Stage x” where x is the stage of treatment at which the IPR for the selected tooth is to occur. The stage at which IPR is to occur may be modified during the MAPS 78 stage of the flowchart 66, which is generally described with reference to FIGS. 4-31 below. Other exemplary commands 82, 112 shown in FIG. 3 include “Relative Intrusion,” “Curve of Spee Flattening,” and “Class II Deepbite.”

With continued reference to FIG. 3, any or all protocols 112 described above or others may be shared via the orthodontic treatment planning and manufacturing system 10 with other orthodontists at 140 in the store 120. The store 120 is accessible by other orthodontists using the orthodontic treatment planning and manufacturing system 10. In FIG. 3, commands 112 in the store 120 may be shared on other computers 12′ and 12″ and so may be displayed in the respective stores at those computers. As an example with reference to FIG. 3, Dr. Smith shares the protocol “Curve of Spee Flattening” 112 and Dr. Williams shares the protocol “Class II Deepbite” 112 with other orthodontists who have access to the store 120. As an example, Dr. Smith can review Dr. Williams' Class II Deepbite command and like it, comment on it, save it, and share it at 142. Similarly, Dr. Williams can review Dr. Smith's Curve of Spee Flattening command and comment on it, like it, and share it.

As is also shown, the store 120 may provide treatment efficacy information concerning the shared protocols 112. In that regard, the store 120 may provide a forum by which multiple offices 12, 12′, 12″ (FIG. 1A) may be connected for sharing protocols 112 and prescriptions 62 between a plurality of orthodontists. For example, for Dr. Smith's command 112, the store 120 indicates a “created on” date and provides additional information regarding the “likes”, “views”, and “shares”, at 142, that may provide some peer-reviewed indication of the efficacy of Dr. Smith's shared protocol 112. If the orthodontist 18 desires to incorporate Dr. Smith's “Curve of Spee Flattening,” the orthodontist 18 may save it at 140 (FIG. 3) to their prescription block 80 for use in their practice.

The orthodontic treatment planning and manufacturing system 10 may record in a database each protocol 112 and/or prescription 62 across all users of the orthodontic treatment planning and manufacturing system 10 and the number of times that command has been used in a treatment plan. Thus, the orthodontic treatment planning and manufacturing system 10 may record information sufficient to develop quantitative information on command usage, protocol development, and prescriptions. In this way, for example, the system 10 may track the most-used commands, the most “likes,” “views,” “saves,” and “shares” of protocol 112 and the most “likes,” “views,” and “shares” of prescriptions so that it is possible to determine which orthodontists are most influential with regard to one or more of the number of likes, views, saves, and shares within the orthodontic treatment planning and manufacturing system 10. Additionally, protocol 112 saved can be used to define clinical preferences for a specific user. Those clinical preferences may be general guidelines/rules to be universally applied to different patients for the specific user, and perhaps others who may adopt those clinical preferences.

As shown in FIG. 3, in one embodiment, the orthodontist 18 arranges the commands 82, 112 in an intentional manner to define a treatment protocol portion of the prescription 62. In the exemplary embodiment, the prescription 62 is a listing of single ones of the commands 82, 112 with each command 82, 112 occupying its own line so that a treatment protocol portion of the prescription is defined by a predetermined relative arrangement of the commands 82, 112 in the prescription 62, similar to execution of programing code. As such, the prescription 62 shown in FIG. 3 includes a built-in treatment protocol. In FIG. 3, for example, the built-in treatment protocol may be determined by a top-to-bottom order of the commands 82, 112 in the prescription 62. Each command 82, 112 defines a single line in the prescription 62. A line-by-line, top-to-bottom order of intended execution of the commands 82, 112 in the prescription 62 is the built-in treatment protocol. Thus, in the example, the treatment protocol of the prescription 62 is the following order: Crown Torque 98 and then Rotate 96 for the sequence of movement of the teeth 90. In this way, according to one embodiment, the orthodontist 18 arranges the commands 82, 112 line-by-line in a column. The line-by-line columnar arrangement is understood to define a hierarchy in the order of application between the listed commands 82, 112. The hierarchy determines which of the commands 82, 112 is to be executed first, second, third, and so on. In FIG. 3, the orthodontist can then save (i.e., “Save As”) that prescription 62 to a protocol 112 named Upper Arch Development.

In view of the above, embodiments of the invention provide a treatment protocol of the prescription 62 for orthodontic treatment of the patient 30 for all or a portion of teeth movement from T1 to at or near T2. This may include only the built-in treatment protocol, described above with respect to a line-by-line order, but may include the built-in treatment protocol in combination with other treatment protocols with other commands 82, 112. Specifically, for example, and with reference to the exemplary prescription 62 of FIG. 3, while the prescription 62 includes Upper Arch Development command 112, in which there is a built-in treatment protocol, embodiments of the invention may also specify an order of commands 82, 112 applied to teeth other than the upper anterior teeth 90 or to the upper anterior teeth 90 at a different time during orthodontic treatment. Stated another way, application of a treatment protocol of commands 82, 112 to teeth other than the upper anterior teeth 90 may be at times before, during, and after Upper Arch Development command 112 is applied to the upper anterior teeth 90. In this way, embodiments of the invention facilitate building a treatment plan to address the patient's diagnosis 32 by incorporating specific treatment protocols of the commands 82, 112 into the prescription 62 to efficiently address the patient's malocclusion. The system 10 may automatically determine an order of application of the commands 82, 112. Embodiments of the orthodontic treatment planning and manufacturing system 10 disclosed herein provide the prescription 62 in which a treatment protocol or a portion of the treatment protocol is specified and from which a treatment plan is more efficiently constructed. The treatment plan may be aligned with the intent of the orthodontist 18 from the outset of treatment. Advantageously, this reduces the time between diagnosis 32 and orthodontic treatment of the patient 30.

To those ends and with reference to FIGS. 3, 4A, and 4B, in an exemplary embodiment, the clinician may assemble the treatment protocol or a portion of the treatment protocol of the prescription 62 in the MAPS 78 section of the flowchart 66. That is, the orthodontic treatment planning and manufacturing system 10 permits arrangement of the time and rearrangement of the time at which any single one or combination of commands 82, 112 is applied to any selected one or group of the patient's teeth. The orthodontist 18 may arrange the commands 82, 112 during building of the prescription 62 and during MAPS 78 section of the flowchart 66, described below.

In general, in MAPS 78, for example, the commands 82, 112 included in the prescription 62 may be rearranged with respect to one another to revise a previously established built-in treatment protocol for the prescription 62. Also, in MAPS 78, additional commands 82, 112 may be added to the prescription 62 with the treatment protocol established as to the additional commands 82, 112 relative to any previously established commands 82, 112 in the prescription 62. It is also possible for the clinician to remove commands 82, 112 from the prescription 62. In summary, in MAPS 78, the orthodontist 18 may revise the treatment protocol for the prescription 62, may create an entirely new treatment protocol for the prescription 62, or add onto the prescription 62 by specifying additional commands 82, 112 and additional treatment protocol.

To any single one or a combination of those ends, by the exemplary user interface 64 of the orthodontic treatment planning and manufacturing system 10 shown in FIGS. 4A and 4B, the orthodontist 18 may add detail to the prescription 62, specifically, the orthodontist 18 may create or further expand an existing treatment protocol portion of the prescription 62. For example, from FIG. 3, the Upper Arch Development command 112 may be saved in the My Commands and Protocols 80 and so Upper Arch Development command 112 is available in the MAPS 78 section. The My Commands and Protocols 80 is shown in FIG. 3 and is shown as a “My Protocols” block separate from the commands 84 block in a sidebar menu 124 on the left side of FIG. 4A. Separating the commands 112 from the commands 82 eases categorization of these two types of commands 82, 112 for selection by the orthodontist.

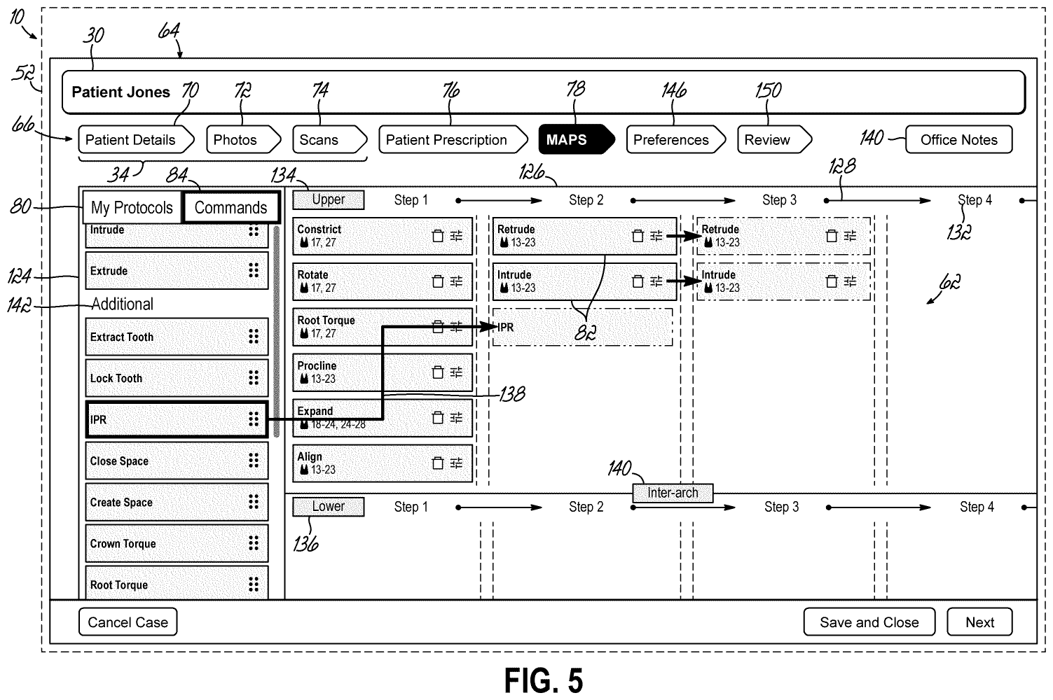

Further in the exemplary embodiment of FIG. 4A, in the sidebar menu 124, the My Protocols block 80 may be automatically populated from the Commands library 84 with protocols 112 usable by the orthodontist. The My Protocols block 80 and the command library 84 are alternatively selectable. Selecting “Commands,” that is, the library 84 (see, e.g., FIG. 5, displays all available commands 82 and protocols 112 in the sidebar menu 124. In that regard, the commands 82, 112 in the My Protocols block 80 are fewer in number than the number of commands 82, 112 available in the library 84.

In MAPS 78, and with reference to FIG. 4A, the orthodontist 18 may select the Upper Arch Development command 112 developed in the user interface 64 of FIG. 3 from the sidebar menu 124 and drag it from the sidebar menu 124 to an adjacent main window 126 (shown by arrow 130). Once released in the main window 126, for example, under “Step 1,” the Upper Arch Development command 112 expands into the constituent commands 82 (i.e., Constrict, Rotate, and Crown Torque) and in the same top-to-bottom order created by the orthodontist 18 and shown in FIG. 3. However, while there may be an order implied by the arrangement of commands 82 in the window 126 in Step 1, the commands 82 in Step 1 may be applied simultaneously to the patient's teeth.

Further in that regard, in the exemplary embodiment, the main window 126 illustrates a time component or period 128 of the treatment protocol. In the embodiment shown, the time period 128 is indicated by a plurality of individual time portions, referred to as “Steps” 132 herein with spaced apart arrows, such as Step 1, Step 2, Step 3, etc. in an x-direction from left to right on the main window 126 of the interface 64. Each Step 132 may specify when any command 82, 112 is to be applied during a treatment period relative to other commands 82, 112 and steps 132. As an example, the treatment period may span a time period required to move the patient's teeth from T1 to at or near T2. The sum of the individual time periods 128, such as the steps 132, may be equal to the treatment period but embodiments of the invention are not limited to equality between the sum of all steps 132 and the treatment period. For example, each step 132 of the treatment protocol may be a time period that is less than the treatment period. Further, the sum of the steps 132 may be less than the treatment period. Therefore, if the treatment period is 12 months, for example, for planned tooth movement from T1 to at or near T2, the steps 132 for application of a selected command 82, 112 may be a time period less than 12 months.

With reference to FIGS. 4A and 4B, steps 132 are further described with reference to treatment time. As shown, the orthodontist 18 may define the prescription 62 as having selected commands 82, 112 executed over with individual time periods, for example, one or more steps 132. In the exemplary prescription 62, four steps 132 are shown. The steps 132 generally represent increasing time 128 from the left to the right on the user interface 64. Step 1 is first, Step 2 is second, and so on with time. With reference to a treatment time, at the start of Step 1, the patient's teeth are at T1, and the patient's teeth are at or near T2 at the end of Step 2.

As is generally shown by the relative time dimension (left to right dimension between dashed lines), each step 132 may define a duration in time relative to the entire orthodontic treatment period. That is, where step 1 through step 4 is the entire orthodontic treatment period, each step 132 is an undivided portion of that entire time. In one embodiment, a sum of the duration of each step 132 equals the treatment period. With respect to one another, the individual steps 132 may be equal in duration (i.e., equal left to right dimension) or may be unequal in duration (i.e., unequal left to right dimensions). For example, if the durations are unequal, Step 1 may last longer in real time than Step 2, or Step 2 may last longer than Step 1. By way of example only, Step 1 of the prescription 62 may last 4 months whereas Step 2 may last 6 months. Embodiments of the invention are not limited to any relative time duration of the steps 132.

More specifically, the steps 132 specify a relative order in time 128 for execution of the commands 82, 112 during the treatment period. For example, commands 82, 112 are placed in Step 1 and are executed first, commands 82, 112 are placed in Step 2 and are executed following execution of commands 82, 112 in Step 1, etc. In the exemplary embodiment shown, each of the Constrict, Rotate, Crown Torque, Expand, and Align commands 82 each occur in Step 1 and in Step 2. Therefore, if orthodontic treatment consists of two steps (i.e., Step 1 and Step 2), each of these commands 82 occurs during the entirety of orthodontic treatment. While the number of steps 132 in the prescription 62 is four, i.e., Step 1, Step 2, Step 3, and Step 4, the number is not particularly limited to any predefined number of steps or relative time per step 132.

With further reference to the example depicted in FIG. 4A, time periods 128 include Step 1 and Step 2. Placement of a command in Step 1 sets Step 1 as an initial time component of the treatment period. As shown, the Constrict command 82 is placed in Step 1. The Procline command 82 is also placed in Step 1 but the Procline command 82 is absent from Step 2. The Procline command 82 therefore occurs only during Step 1. In Step 2, the Retrude command 82 and Intrude command 82 are applied. These commands 82 are not in Step 1. If there is a built-in treatment protocol to the commands 82, in Step 1, the Procline command 82 occurs after the Align command 82 and before the Constrict command 82 in Step 2. The Retrude command 82 follows each of the Constrict through Align commands 82 and the Intrude command 82 follows the Retrude command 82. However, in FIG. 4A, the treatment protocol of the prescription 62 is commands 82 of Step 1 and then commands 82 of Step 2. In other words, each of Constrict, Rotate, Crown Torque, Expand, Align, and Procline commands 82 are simultaneously applied during Step 1. Then, at the completion of Step 1, each of Constrict, Rotate, Crown Torque, Expand, Align, Retrude, and Intrude is simultaneously applied during Step 2. In other words, the commands 82 in Step 1 are applied in parallel with one another, and the commands 82 in Step 2 are applied in parallel with one another.

With reference to FIGS. 4A and 4B, in one embodiment, the main window 126 may visually separate the prescription 62 of commands 82, 112 and steps 132, between an upper arch 134 (“Upper” in FIGS. 4A and 4B) and a lower arch 136 (“Lower” in FIGS. 4A and 4B). Commands 82, 112 positioned in the upper arch 134 are applied to the patient's upper arch (i.e., in the maxillary jaw). Similarly, commands 82, 112 positioned in the lower arch 136 are applied to the patient's lower arch (i.e., in the mandibular jaw). In one embodiment, the main window 126 includes an inter-arch location 140 (“Inter-arch”) to receive commands operational between the upper arch 134 and the lower arch 136. Commands 82, 112 positioned in the inter-arch location 140 may operate between the patient's lower arch and the patient's upper arch. In addition or alternatively, commands 82, 112 positioned in the inter-arch location 140 may operate on one of the upper arch and the lower arch or on each of the upper arch and lower arch.

Also shown in the exemplary embodiment of FIGS. 4A and 4B, when the My Protocols block 80 is selected, the sidebar menu 124 displays a predetermined subgroup 142 of all the commands 112. Those predetermined subgroups 142 may be organized based some commonality between the commands 112. As an example, organization may be (i) based on popularity of the commands 112 across all offices 12, 12′, 12″, (ii) based on association with or usable with a particular tooth or region of teeth 90 in the image area 86 (see, e.g., FIG. 3), (iii) based on those commands 112 recently used by the orthodontist 18, and (iv) based on commands 112 recommended by or available from an appliance manufacturer. Exemplary subgroups 142 are shown in FIGS. 4A and 4B in which commands 112 are grouped together based on a “Recently used” subgroup 144, a “Favorites” subgroup 146, and a “Spark Collection” subgroup 150 within the My protocols block 80 of sidebar menu 124. The commands 112 from any single one of the subgroups 142 in the My Protocols block 80 are available for selection and inclusion in the main window 126.

Additionally, or alternatively, the orthodontist 18 may directly access the library 84 at any time by selecting the “Commands” library 84 by which a complete library of commands 82, 112 is available in the sidebar menu 124. In one embodiment, the library 84 contains all commands 82, 112 readily available to the clinician. While exemplary subgroups 142 are shown, embodiments of the invention are not limited to any particular methodology for grouping of the commands 82 and for grouping of the protocol 112 to any subgroup 142 for display. In FIG. 5, for example, only commands 82 that are universal for treatment are shown. However, the selectable commands 82, 112 displayed in the protocol block 80 should facilitate efficient assembly and/or revision of/addition to the prescription 62 in the main window 126.

Referring to FIG. 4B, any single one of the subgroups 142, such as the Spark Collection subgroup 150, may include one or more of the commands 112 defined by another user or the orthodontist internally to the orthodontic treatment planning and manufacturing system 10. As an example, the commands 112 may include those protocols constructed by technical professionals at, for example, Ormco Corporation (i.e., “by the Spark Team,” see, e.g., Severe Crowding command 152 in FIG. 4A and FIG. 4B), and may be the result of clinical prescription testing of large numbers of patients. These commands 112 may also be referred to as system commands 154 and are listed in at least the subgroup 150. They may also be listed in other subgroups 142, for example, in the Recently used subgroup 144, if they satisfy the commonality for automatically being selected for that subgroup 142. Although not shown, the subgroup 150 (“Spark Collection”) may also list commands 82 in addition or as an alternative to commands 112. Unlike some of the other commands 82, 112, the system commands 154 may not be modifiable directly by the orthodontist 18 or technician 26. However, the orthodontic treatment planning and manufacturing system 10 may permit submission of suggested modifications or permit individual clinicians to prepare protocols 112 incorporating selected ones of the system commands 152. Stated another way, the orthodontic treatment planning and manufacturing system 10 may permit building of protocols 112 incorporating the system commands 152. With this capability, clinicians may develop protocols 112 incorporating system commands 152 and if an appliance manufacturer or a group of clinicians finds that the specific protocol 112 is effective, the system 10 may permit elevation of the specific protocol 112 to a level of the system command 152. For example, in FIGS. 4A and 4B, the “Upper Arch Development” command 112 may be a protocol that, after its efficacy is established, is promoted by the appliance manufacturer in the subgroup 150 and may ultimately be designated as a system command 152.

With reference to FIG. 4B, the main window 126 is shifted to display the lower arch 136 whereas detail of the upper arch 134 is shown in FIG. 4A. In FIG. 4B, application of the Lower Curve of Spee Flattening command 112 to the lower arch 136 is shown. As an example, the Lower Curve of Spee Flattening command 112 may be selected in the Spark Collection subgroup 150 and moved to Step 1 of the main window 126. Similar to the Upper Arch Development command 112 in FIG. 4A, the Lower Curve of Spee Flattening command 112 expands into the constituent commands 82 that define the Lower Curve of Spee Flattening command 112 in the top-to-bottom order defined during building the Lower Curve of Spee Flattening command 112. In this case, individual ones of each of Constrict, Rotate and Root Torque commands 82 define the Lower Curve of Spee Flattening command 112 and are shown expanded in the main window 126. Also shown, the Lower Curve of Spee Flattening command 112 is placed in both Step 1 and in Step 2. Again, if the treatment protocol portion includes 2 steps 132, then the Lower Curve of Spee Flattening command 112 occupies both steps 132 (i.e., Step 1 and Step 2) and is applicable during treatment from T1 to at or near T2.

Also in the lower arch 136, additional commands 82 are prescribed in each of Step 1 and Step 2. The commands 82 in addition to the Lower Curve of Spee Flattening command 112 are Expand, Align, Procline, Retrude, and Intrude with each of Expand and Align commands 82 being in Step 1 and Step 2, Procline being in Step 1 only, and Retrude and Intrude being in Step 2 only. In this case then during addition to the prescription 62, for example, the Procline command 82 may be selected from the library 84 and placed in the Step 1 below the Align command 82. The Expand command 82 and the Align command 82 may each be selected from the library 84 and placed in each of Step 1 and Step 2. While the relative visual position of the Procline command 82 in the main window 126 may suggest that the Procline command 82 is active in a time period after the Align command 82 in Step 1, according to embodiments of the invention, the Procline command 82 is applied in parallel, that is, simultaneous with the Lower Curve of Spee Flattening and the Align command 82. Other arrangements of commands 82 are possible, embodiments of the invention are not limited to the arrangements shown. The order and placement of any command 82, 112 in any of the Steps 132 may be changed as is described further below with respect to FIG. 5.

According to one embodiment, addition to and/or rearrangement of commands 82, 112 in the main window 126 is further illustrated in FIG. 5. Commands 82, 112 may be added to the prescription 62 in the main window 126 at any particular step 132. For example, the orthodontist 18 may add a command to be applied at either or both of Step 1 and Step 2. To do so, the orthodontist 18 may select the My Protocols 80 or the library 84 at “Commands” (as shown). From the library 84, the orthodontist 18 may select the IPR command 82 and drag it to the main window 126. This is shown by arrow 138. In this example, the orthodontist 18 places the IPR command 82 in Step 2. The IPR command 82 may be positioned anywhere in the main window 126 and be added to the prescription 62. For example, the IPR command 82 may be placed in Step 2 after the Intrude command 82.

Addition of a command 82, 112 may also include simultaneous rearrangement of the commands 82, 112. For example, with continued reference to FIG. 5, the orthodontist 18 may position the IPR command 82 between two existing commands 82, such as Retrude command 82 and Intrude command 82 at the top of Step 2. When the IPR command 82 is so positioned, the orthodontic treatment planning and manufacturing system 10 may move Intrude command 82 and Retrude command 82 to Step 3. This movement is schematically shown in phantom line with the Intrude command 82 and Retrude command 82 moved to the right to Step 3. The IPR command 82 is a chairside procedure that does not span across steps 132. In other words, it does not occupy time within the system 10. Conceptually, it is a pause in the treatment protocol. For these types of commands, when added to the treatment protocol, their addition may automatically shift any movement commands 82 in the Step to the next Step.

In addition or as an alternative to automatic step movement, the system 10 may also rearrange commands within a step. For example, when tooth movement commands 82 are added to the window 126, they may cause the system 10 to shift existing commands 82 in the Step downward. In this way, the orthodontist 18 may add commands 82 while also rearranging the commands 82 in any single one of the steps 132 or between steps 132. While not shown, the orthodontist 18 may add commands 112 to the main window 126 by the same process. The orthodontist 18 may also rearrange any commands 82 in the main window 126 by selecting the command 82 and dragging the command to a new location in the step 132 or to another step 132. The same methodology may be utilized to add, remove, or rearrange commands 82, 112 from the lower arch 136.

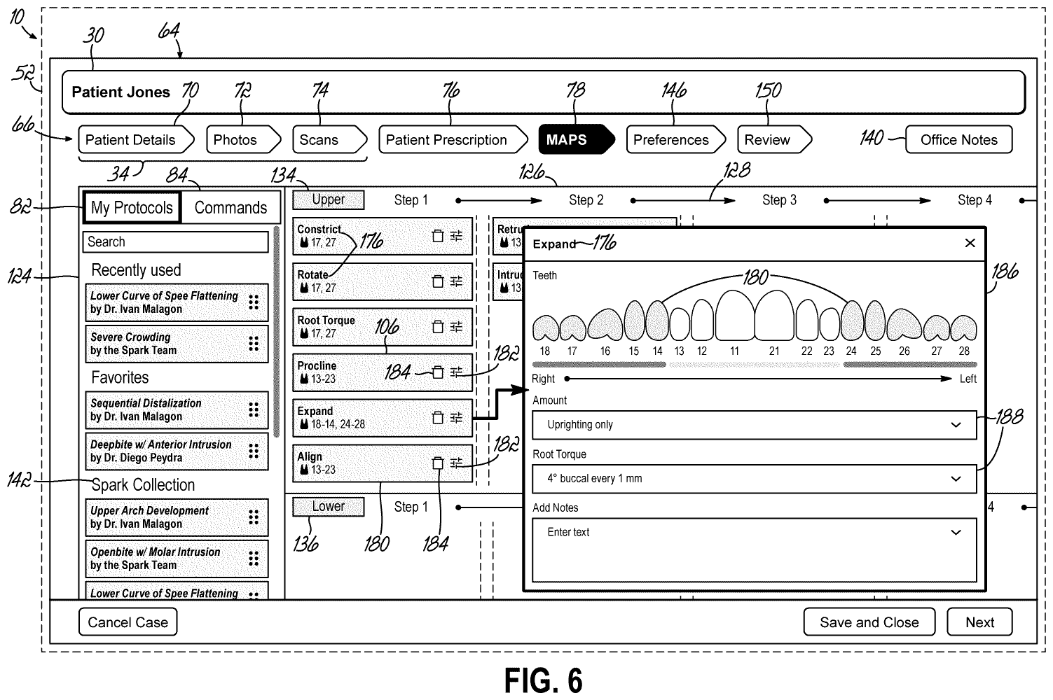

Referring to FIG. 6, in one exemplary embodiment, one or more of the commands 82, 112 may include additional operational information, which may be include command parameters, within an encircling border 106. In the examples shown, the border 106 is rectangular and so may be referred to as a block. With reference to construction of the prescription, the blocks are visually arranged in columns and/or rows. As shown, the border 106 circumscribes the one, two, or three-word instructions for orthodontic treatment. The command 82, 112 may be referred to as a block. As examples, the command parameters may include any single one or a combination of a command identifier 176, the targeted teeth 180, details 182, and a removal feature 184 to name a few. With reference to the Expand command 82, the command parameters indicates that the teeth “16-14” and “24-28” are targeted teeth 180 for the Expand command 82. Selection of the details 182 opens a details window 186. The details window 186 provides predetermined command parameter information for the given command 82. These parameters may be user modified so as to customize the selected command 82 to the patient 30. The details window 186 may replace or augment the command parameters 100 described with reference to the exemplary embodiment of FIG. 3.

In FIG. 6, for the Expand command 82, the targeted teeth 180 may be selected according to a visual representation of teeth for the upper arch 134. Further, the visual representation of human teeth may change color or shading in accordance with the selected targeted teeth 180 in the details window 186. As shown, the targeted teeth 180 “18-14” and “24-28” as indicated are shown shaded relative to non-targeted teeth 13-23 in the details window 186. Additional selections may include specific details for application of the Expand command 82 to the targeted teeth 180. As shown, for example, the orthodontist 18 may select movement parameters 188, for example, “Amount” and “Root Torque,” for application to the targeted teeth 180. The orthodontist 18 may make additional notes in the “Added Notes” section of the details window 186.

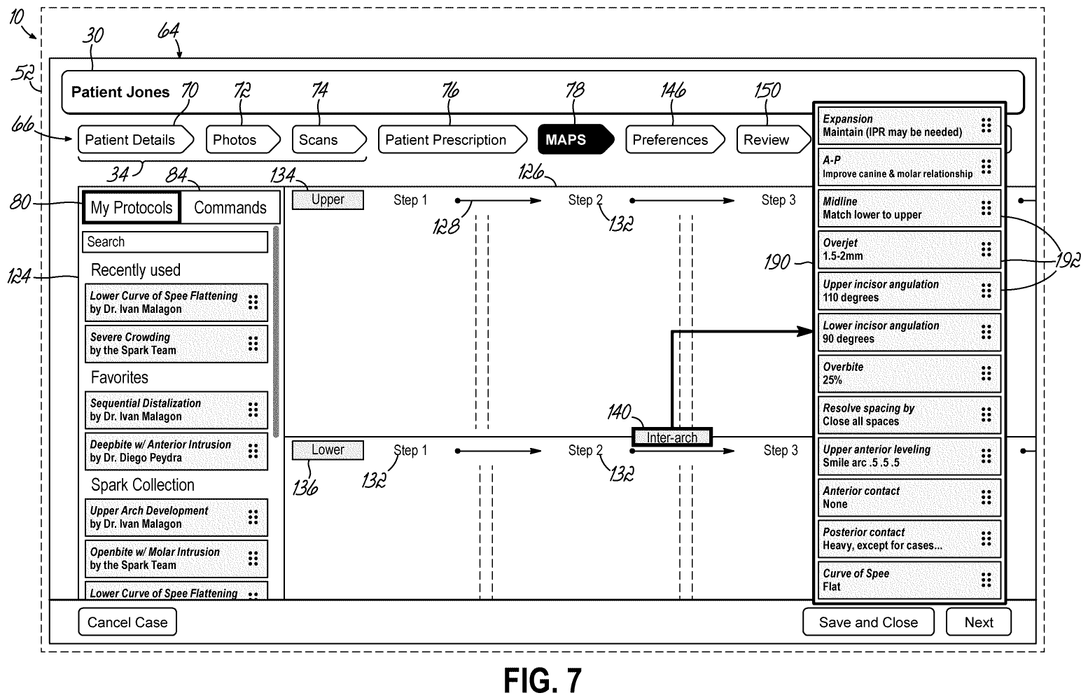

With reference to FIG. 7, in one embodiment, the inter-arch location 140 is visually located and opened in the main window 126 between the upper arch 134 and the lower arch 136. The commands 82 in inter-arch location 140 may be time independent. That is, operation of any inter-arch commands may not be associated with any of the steps 132. The inter-arch location 140 is selectable so as to expand into an inter-arch goal window 190 in which a plurality of inter-arch goals 192 may be selected. Generally, one or more of the inter-arch goals 192 may provide an indication that orthodontic treatment has reached its completion. Stated another way, the orthodontist 18 may prescribe conditions between the upper arch 134 and the lower arch 136 for when at least a portion of treatment has reached a conclusion. For example, the orthodontist 18 may prescribe that “Overjet” be within a specific measurement as an indication that proper occlusion has been achieved. Although this may indicate that T2 has been reached, embodiments of the present invention are not limited to any single one of the inter-arch goals 192 as being the sole indicator that actual treatment has reached T2. Rather, in one embodiment, when all the inter-arch goals 192 are reached, orthodontic treatment is concluded.

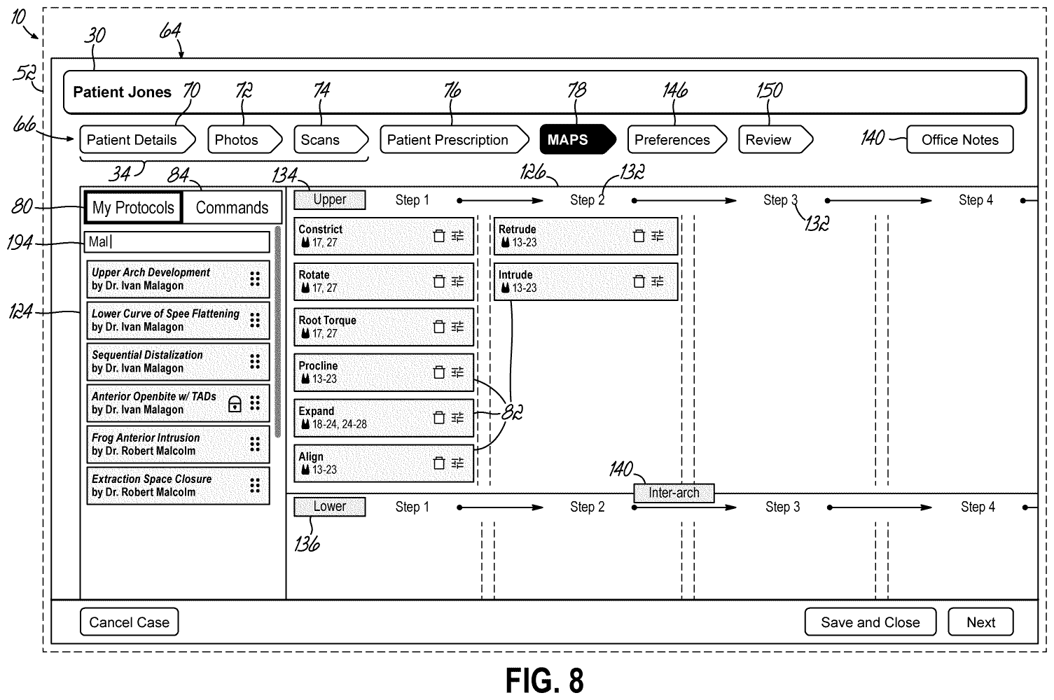

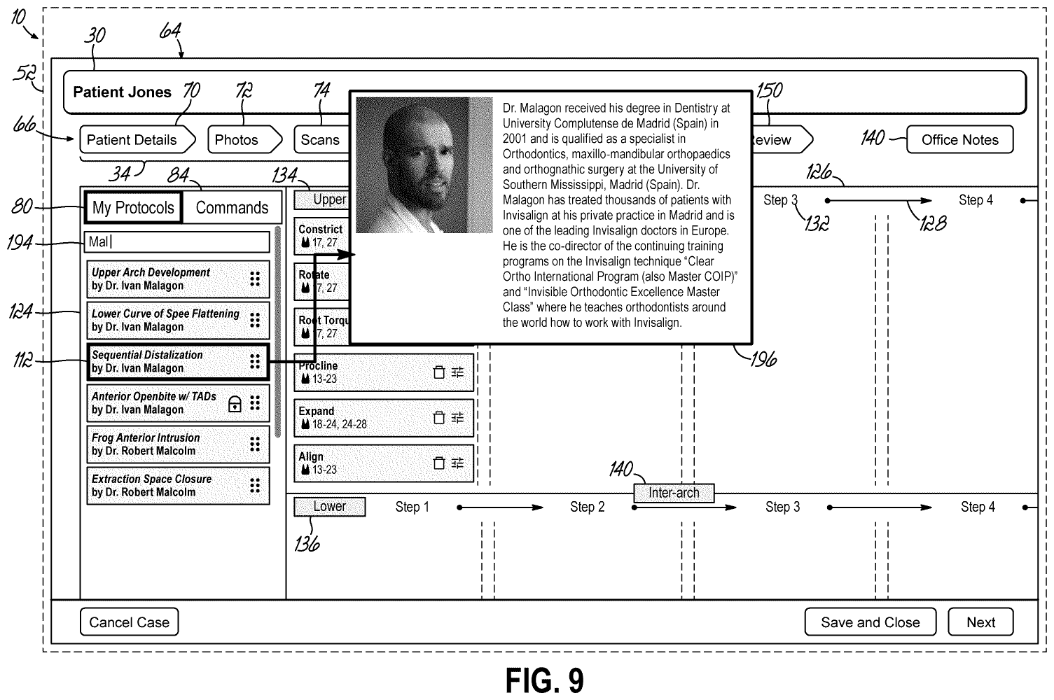

With reference to FIGS. 8 and 9, in one embodiment, the user interface 64 includes a search feature 194 by which the clinician may search for one or more commands 82, 112 or specific text within commands 82, 112 in My Protocols 80 or in the library 84. For example, if the clinician wishes to locate protocols 112 of a particular orthodontist, the clinician may search for protocols 112 built by the specific orthodontist. In the example shown, the clinician may search for Dr. Ivan Malagon by entering the text “Mal” into the search feature 194. The orthodontic treatment planning and manufacturing system 10 will then populate the sidebar menu 124 with commands in which “Mal” appears in the block 106. As shown, based on that search, the sidebar menu 124 is populated with protocol 112 by Dr. Ivan Malagon and by Dr. Rober Malcolm. With reference specifically to FIG. 9, in one embodiment, selection of a single one of protocols 112 may provide background and/or other information 196 pertaining to the author of the selected protocol 112, such as Dr. Ivan Malagon, who constructed the Sequential Distalization command 112.

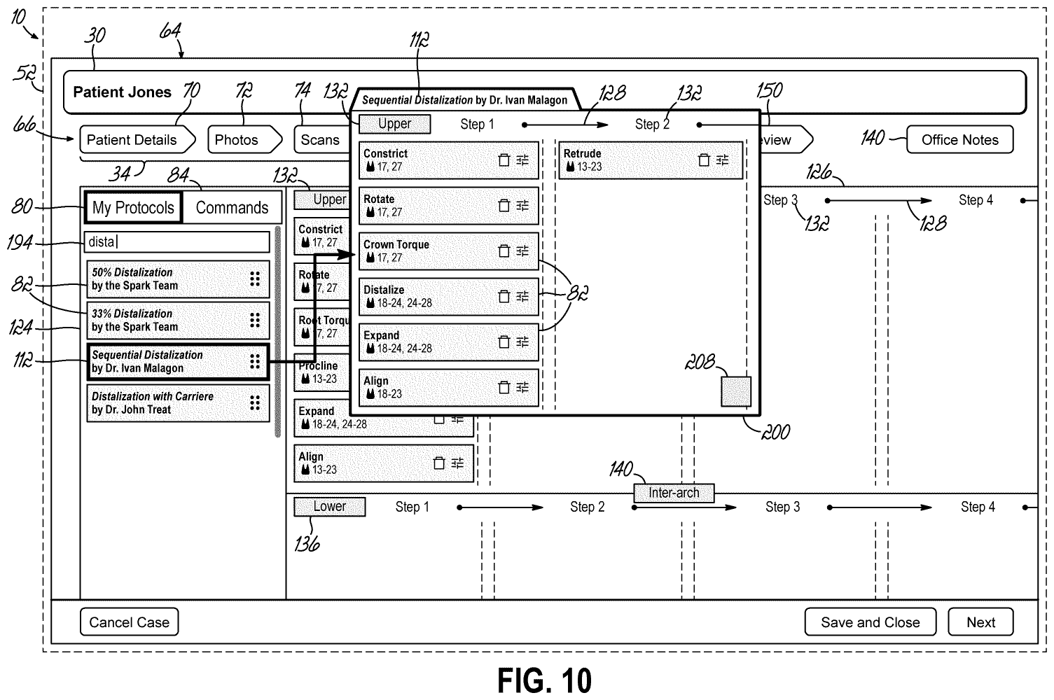

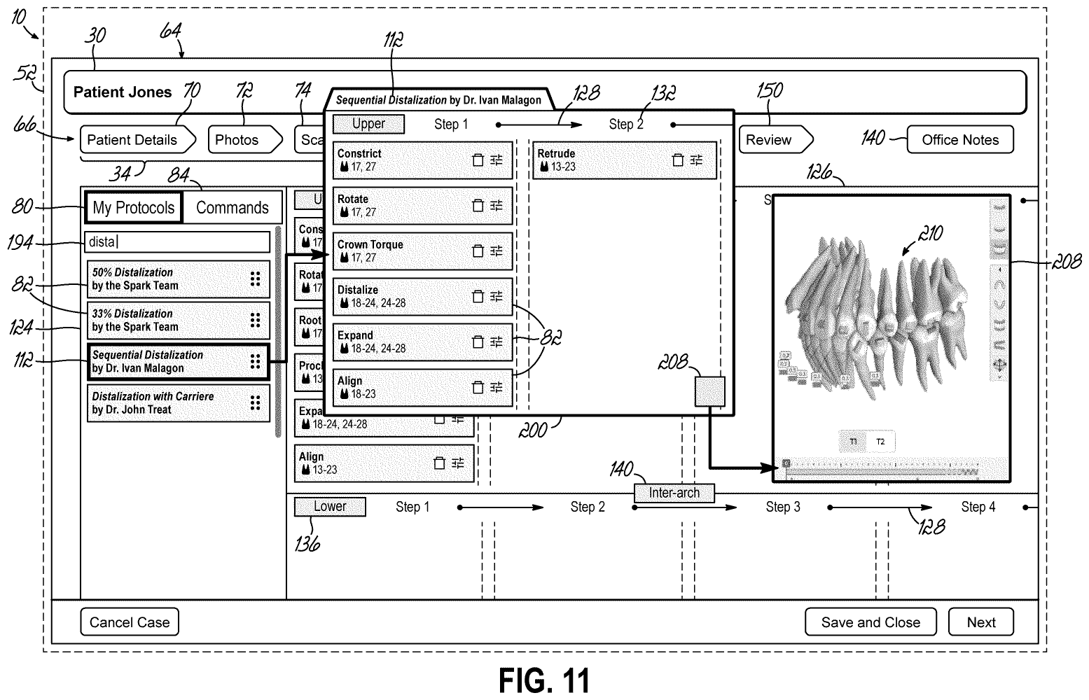

With reference to FIGS. 10 and 11, in one embodiment, the clinician may also implement the search feature 194 to locate commands adapted for a particular tooth movement in the prescription 62. For example, searching for “dista” causes the orthodontic treatment planning and manufacturing system 10 to populate the sidebar menu 124 with blocks 106 in the My Protocols 80 or in the library 84 that are designed to produce distalization. Further, selection of any single one of the commands 82, 112 may momentarily cause an enlarged details window 200 to appear in which the details of the selected protocol 112 are shown. That is, it is not necessary for the clinician to drop the protocol 112 in the main window 126 to see the constituent commands 82 of the protocol 112. In the example shown, selection of the Sequential Distalization command 112 by Dr. Ivan Malagon causes the orthodontic treatment planning and manufacturing system 10 to open the details window 200 for the Sequential Distalization command 112. As shown, the Sequential Distalization command 112 defines a 2-step treatment protocol for the upper arch 134 with 7 commands 82 divided between the 2 steps. If the clinician drags and drops the Sequential Distalization command 112 into the main window 126, for example, the clinician can anticipate that the commands 82 and their arrangement with respect to steps 132 will appear in the main window 126 at the dropped location. In one embodiment and with reference to FIG. 11, the details window 200 further includes a sample movie window 208. When selected, the sample movie window 208 enlarges. The clinician is then able to selectively view a sample movie preview of the selected command 112 (e.g., Sequential Distalization command 112) on a virtual model of teeth 210. The virtual model of teeth 210 may be a virtual model of the patient's teeth or a generic model of teeth. The clinician may play the movie to watch the intended effect of the protocol 112 on teeth from T1 to T2 or any shorter time period from T1 to T2 or over defined Steps 132 indicated in the details window 200. This may enable the clinician to make a determination as to whether the protocol 112 is applicable to properly fix the malocclusion of the patient 30.

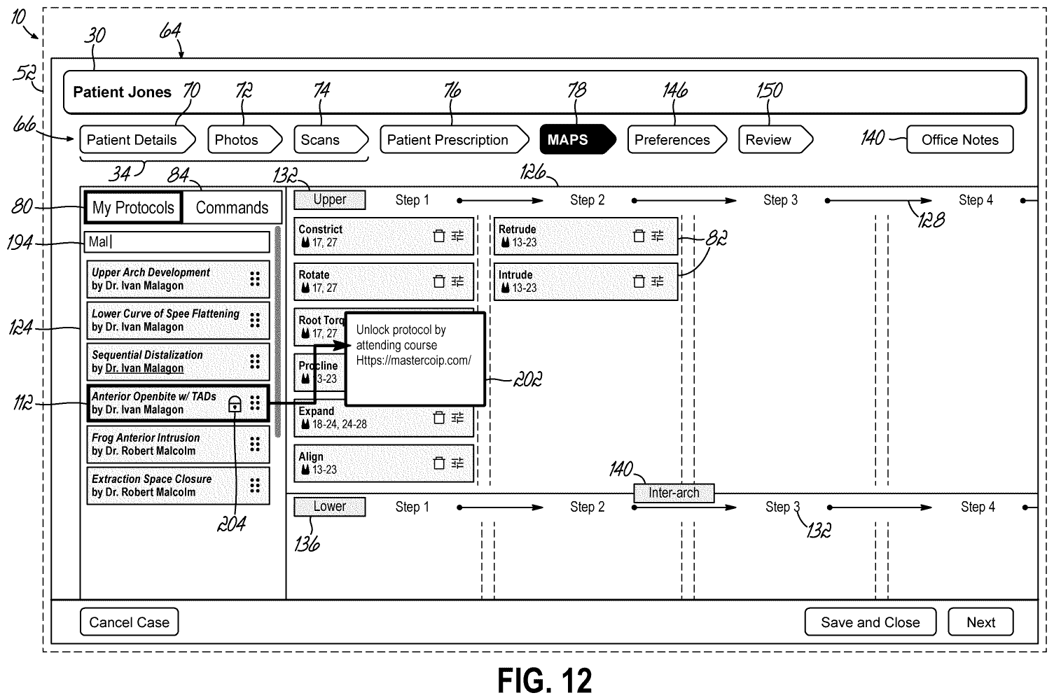

In one embodiment, and with reference to FIG. 12, the orthodontic treatment planning and manufacturing system 10 includes a locking feature by which the system 10 may control whether any single one of commands 82 or 112 is usable or the constituent commands 82 of a protocol command 112 are viewable by the clinician. As an example, if a clinician selects a command 82, 112 in the My Protocols block 80 that the clinician is not yet authorized to apply to the patient 30, a lock window 202 may appear in which the orthodontic treatment planning and manufacturing system 10 provides instructions by which the clinician may unlock the command 82, 112. In the example, the Anterior Openbite w/TADs command 112 by Dr. Ivan Malagon is locked against unauthorized use according to a lock symbol 206. If the Anterior Openbite w/TADs command 112 is selected, the lock window 202 appears, which includes instructions for unlocking the command 112. As shown, unlocking the command 112 requires further education (e.g., a course) after completion of which the clinician may be given an unlock code or the entity that organizes and controls the contents of the library 84 may authorize the clinician internally to orthodontic treatment planning and manufacturing system 10. In any case, once the clinician satisfies the precondition for use of the command 112, the system 10 authorizes the clinician's application of the protocol 112 in the prescription 62 for the patient 30.

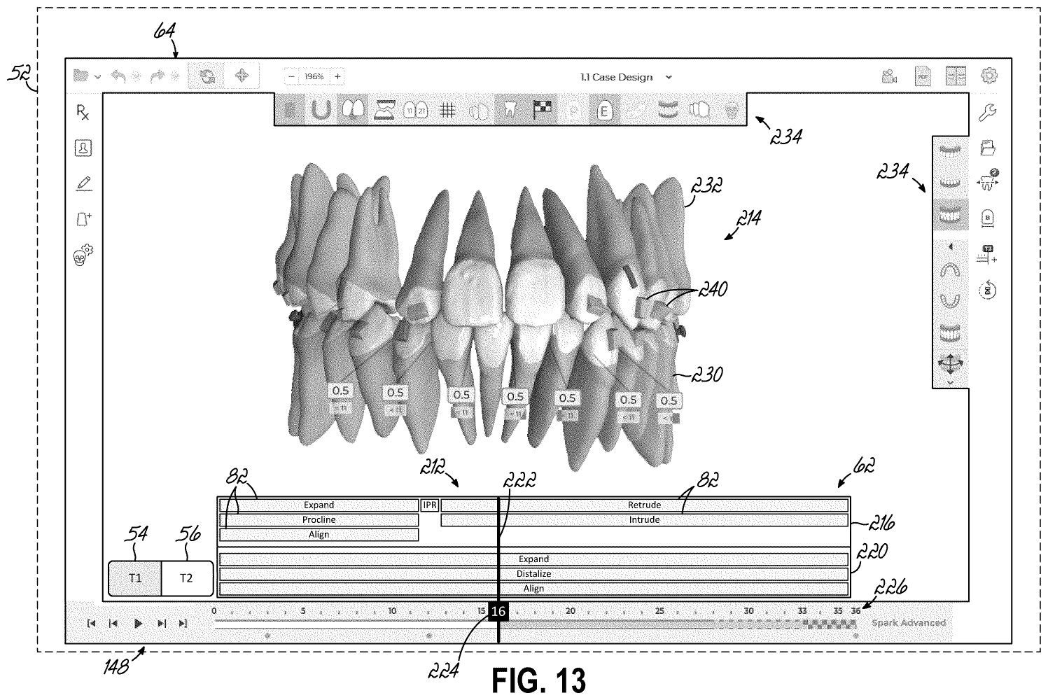

Once the prescription 62 is assembled with commands 82, 112 and treatment protocol, the orthodontic treatment planning and manufacturing system 10 permits the clinician to study virtual application of the prescription 62 to a virtual model of teeth, for example, the 3D digital model 54 (i.e., T1 model for the patient 30). In one embodiment, and with reference to FIGS. 13, 14, and 15, the orthodontic treatment planning and manufacturing system 10 may apply the prescription 62 including the treatment protocol to one or more 3-D digital models 54 of the patient's teeth. As is shown in FIG. 13, an integrated staging chart 212 is shown in conjunction with a 3D model 214 of the patient's teeth. The clinician may view a movie of application of the prescription 62 to the virtual model 214 of the patient's teeth from T1 to T2 by activating the integrated staging chart 212. As shown, the integrated staging chart 212 is divided in an upper and lower charts. In that regard, for example, the chart 212 includes an upper arch chart 216, which includes the commands 82 arranged in steps 132 corresponding to the upper arch 134 in the main window 126, such as that shown in FIG. 4A. The integrated staging chart 212 also includes a lower arch chart 220, which includes commands 82 arranged in steps 132 corresponding to the lower arch 136, such as that shown in FIG. 4B.

In the exemplary embodiment, a slider 222 overlays each of the upper arch chart 216 and the lower arch chart 220 at one stage 224 of a plurality of stages 226. Steps 132 in the prescription 62 may have some correspondence with the stages 226 in the chart 212 and aligners for orthodontic treatment. For example, a single stage 224 as indicated in the system 10 according to the chart 212 may represent one step 132 in the prescription and also one orthodontic appliance, such as an aligner, for one of the upper and lower arches for the patient to wear. Thus, a single step 132 may represent a single stage 224 and, for treatment of the upper and lower arches, may require two aligners, one aligner for the upper arch and one aligner for the lower arch. By contrast, a single step 132 may represent a plurality of stages 224, for example twelve stages 226 (see e.g., FIG. 13), and a plurality of aligners. Further, a single step 132 may require one chairside procedure during one office visit or a plurality of chairside procedures during one office visit.

In view of the step 132 and stage 224 relationship, in FIGS. 13, 14, and 15, overlap of the slider 222 on the commands 82 indicates which of the commands 82 is applied at each stage 224. The integrated staging chart 212 associates the steps 132 shown in FIGS. 4A and 4B with the plurality of stage 226. As is shown in the exemplary embodiment of FIG. 13, the upper arch chart 216 indicates 3 steps in the prescription 62—Step 1: Expand, Procline, Align; Step 2: IPR; and Step 3: Retrude, Intrude. The lower arch chart 220 is a single step of Expand, Distalize, Align. Each of the steps 132 in the upper arch chart 216 and the lower arch chart 220 are associated with one or more of the plurality of stages 226. Thus, the time period 128 of the steps 132 is divided into stages 224 in FIG. 13. The number of stages 224 for any particular step may depend on the magnitude of the tooth movement for the tooth that moves the greatest distance, referred to as the lead tooth. The system 10 may define a maximum permitted movement per stage, for example, 0.25 mm. As an example, the largest magnitude of movement divided by the speed limit determines the minimum number of stages 226 required.

By activating play of a movie at 148 in FIGS. 13, 14, and 15, the slider 222 translates from T1 54 (left) to T2 56 (right) as the system 10 applies the prescription 62 to the virtual model 214. The location of the slider 222 overlays on each of the upper arch chart 216 and the lower arch chart 220 to indicate which commands 82 are active (i.e., applied to the virtual teeth) at any particular time. In other words, in the exemplary embodiment shown, a command is active if the slider 222 intersects it. By studying the virtual tooth movement, the clinician can assess the efficacy of the prescription 62 and revise the prescription 62 as necessary.

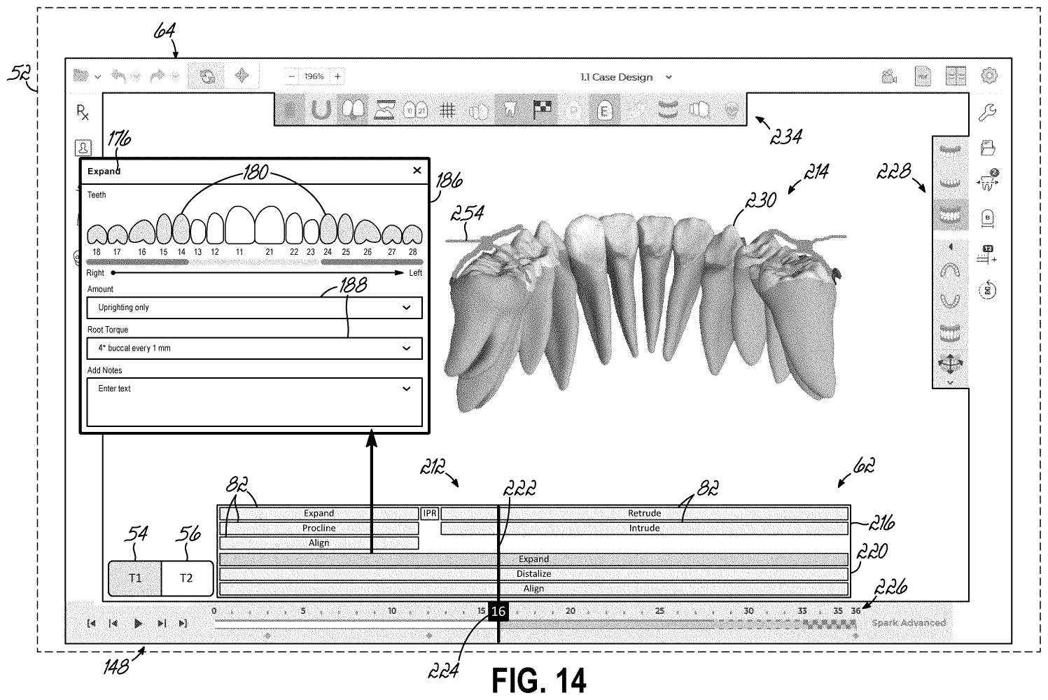

In that regard, and with reference to FIGS. 13, 14, and 15, in one embodiment, revisions to the prescription 62 are possible in the integrated staging chart 212. The clinician may select a single one of the commands 82 in the prescription 62 and edit the selected command 82 within the details window 186 (described above with reference to FIG. 6). The clinician may adjust movement parameters 188, if available, for the command 82. For example, the clinician may select the Expand command 82 in the lower arch chart 220 (FIG. 14). The details window 186 for the Expand command 82 opens, as shown. The clinician may then adjust any of the targeted teeth 180 and/or the movement parameters 188 for the command 82. The effect of the revisions to any of the targeted teeth 180 and/or the movement parameters 188 may be immediately viewable with respect to the virtual model 214 with the slider 222 positioned to overlap the selected, revised command 82. Alternatively, the revised movement parameters 188 are viewable when the clinician plays the movie at 148.

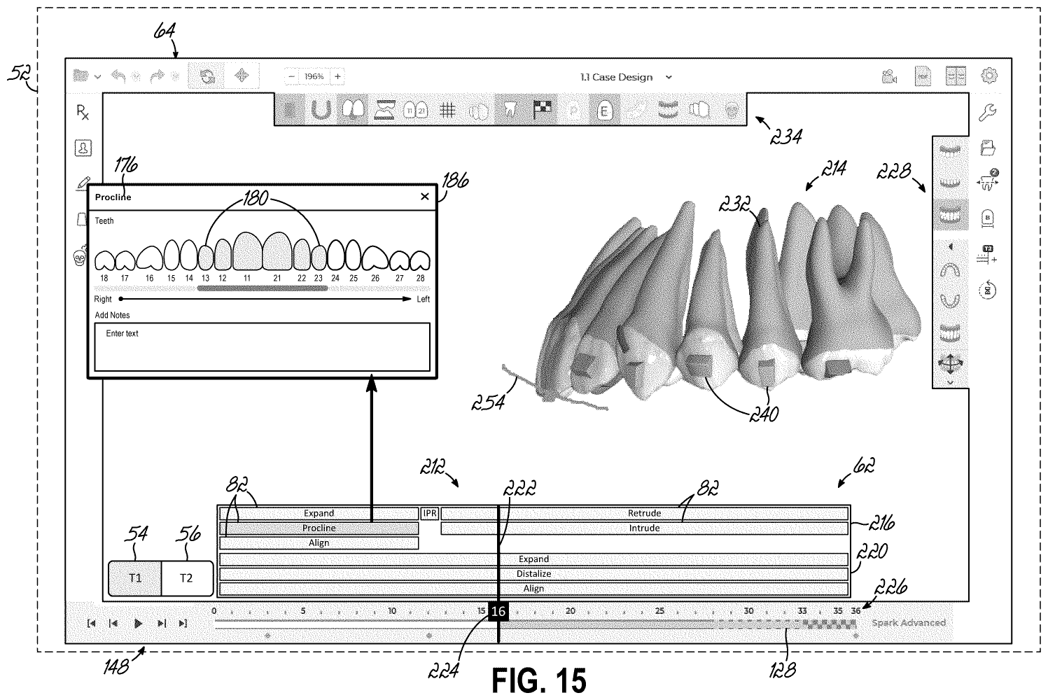

Similarly, as a further example and with reference to FIG. 15, the clinician may select the Procline command 82 in the upper arch chart 216. The details window 186 for the Procline command 82 opens, as shown. The clinician may adjust any of the targeted teeth 180 for the command 82. The effect of the revisions to any of the targeted teeth 180 may be immediately viewable with respect to the virtual model 214. Further, in one embodiment, selection of any single one of the commands 82 from the upper arch chart 216 or the lower arch chart 220 causes the orthodontic treatment planning and manufacturing system 10 to display only the corresponding patient's arch of the virtual model 214. As shown in FIG. 14, a lower arch 230 of the virtual model 214 is shown, and in FIG. 15, the upper arch 232 of the virtual model 214 is shown. In the exemplary user interface 64 shown, various tools 228, 234 are available. Tools 228 are configured to manipulate the virtual model 214 shown in the window. Tools 234 are configured to manipulate relative positions of one or more teeth shown in the arch 230, 232. One exemplary tool 254 in each of FIGS. 14 and 15 permits the clinician to adjust an amount of virtual tooth movement for the command 82 selected. Thus, the tool 254 is associated with a particular command 82, 112 and permits adjustment of any command parameters 188 associated with the command 82, 112. The tool 254 may only appear when the command 82, 112 is selected and permits adjustment of the virtual teeth that are the subject of the command 82, 112.

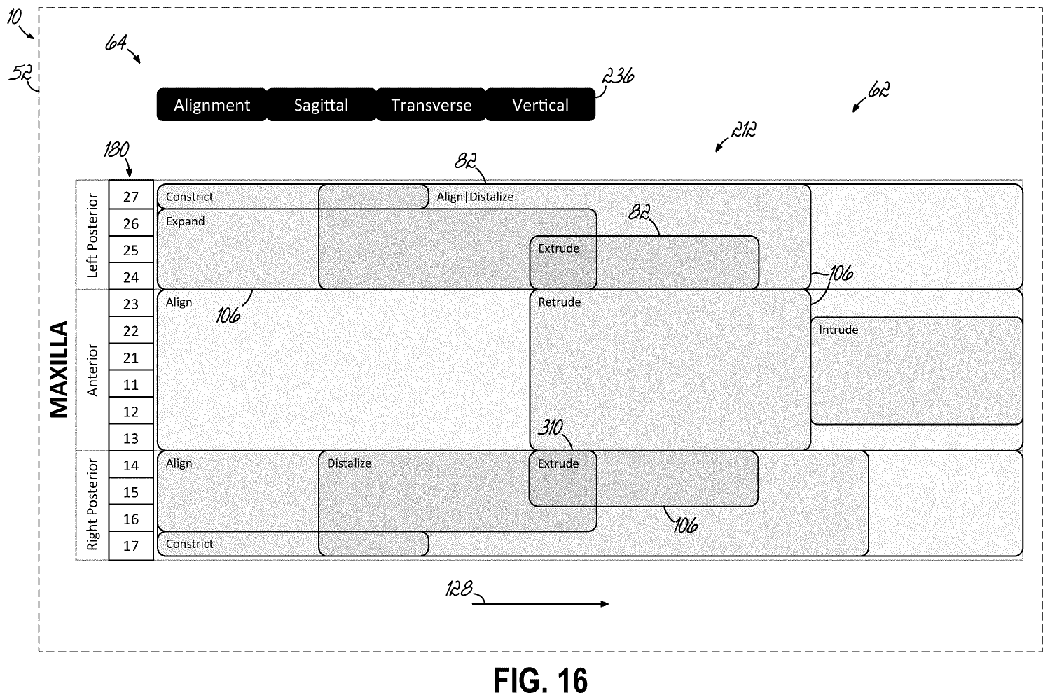

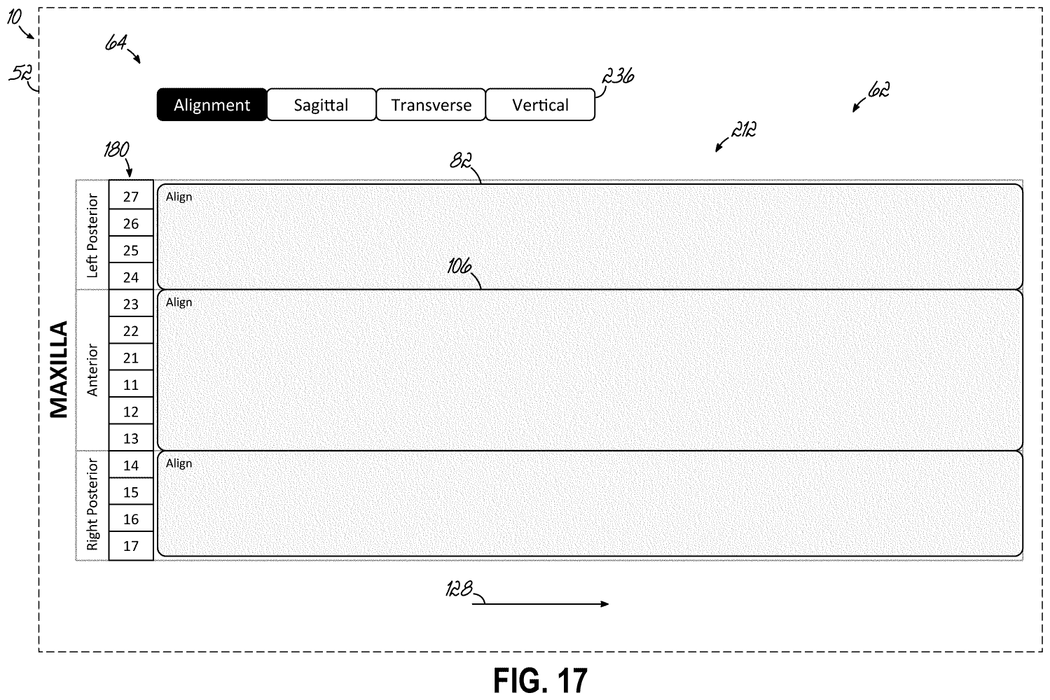

In one exemplary embodiment of the invention, and with reference to FIGS. 16-20, the integrated staging chart 212 may display the prescription 62 with the treatment protocol in a different format from that shown in FIGS. 14 and 15, for example. In FIG. 16, the prescription 62 has overlapping blocks 106 of the commands 82. That is, a boundary that visually defines the block 106 for selected ones of the commands 82 overlap. Conceptually, translucent blocks 106 are stacked one upon another. So, rather than a columnar arrangement shown in FIG. 14 for example, the blocks 106 overlap with reference to the time period 128. By overlapping, the boundaries of multiple block 106 may cross, but each command 82, 112 is visually identifiable relative by its respective boundary. In the areas of overlap, the commands 82, 112 are simultaneously applied to the teeth at that point in time 128. As an example and with reference to FIG. 16, the Align|Expand command 82, the Distalize command 82, and the Extrude command 82 for the maxilla, right posterior 1-17 overlap in time at timeframe 310. The integrated staging chart 212 shown in FIG. 16 provides a visual depiction of layers of commands 82, 112 with respect to each of the targeted teeth 180. Overlapped commands 82 are shown in progressively darker shades. The integrated staging chart 212 in FIG. 16 may be separable into individual command layers. In FIGS. 17-20, embodiments of the system 10 permit each of the layers stacked together in FIG. 16 to be separately depicted.

In that regard, in FIGS. 16-20, in an exemplary embodiment, a layer selection bar 236 is depicted by which the clinician may optionally select which commands 82, 112 are shown in the integrated staging chart 212. The layer selection bar 236 may categorize the commands 82, 112 in the prescription 62 as those directed to “Alignment,” “Sagittal,” “Transverse,” and “Vertical.” Thus, when Alignment is selected in the layer selection bar 236 with all other categories deselected, the integrated staging chart 212 may depict all commands 82 in the prescription 62 categorized under Alignment. This is shown, by way of example only, in FIG. 17 in which the Align command 82 is applied for all targeted teeth 180 for the entirety (per the time period 128) of the prescription 62.

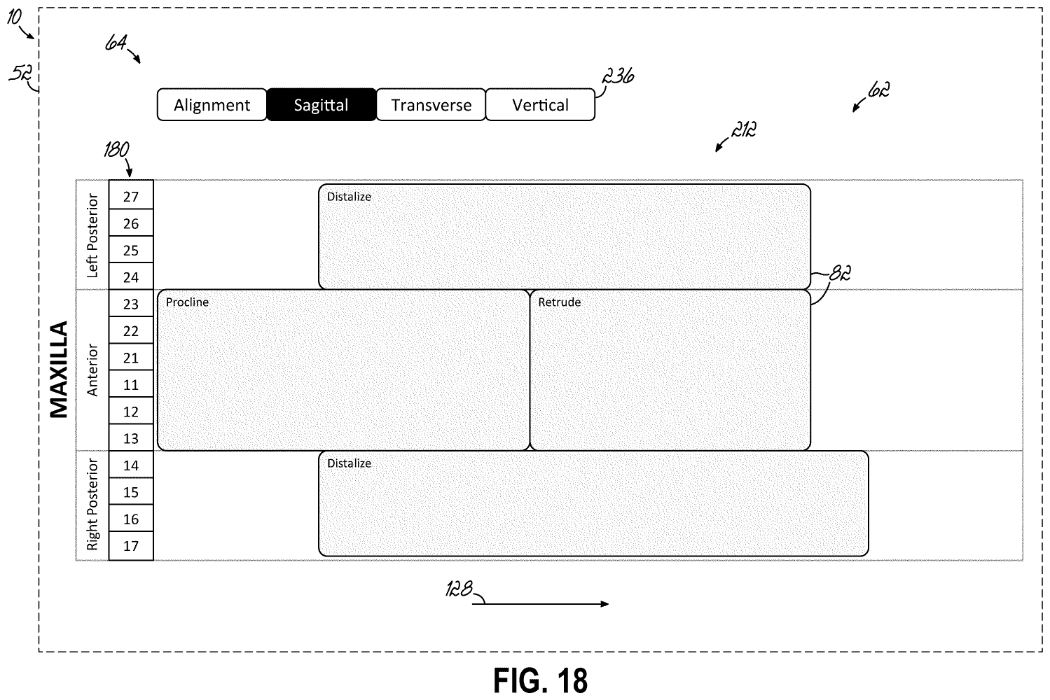

Similarly, and with reference to FIG. 18, when Sagittal is selected from the layer selection bar 236 with all other categories deselected, commands categorized under Sagittal are shown in the integrated staging chart 212. In the example shown, each of

Distalize, Procline, and Retrude commands 82 is displayed. As shown, the Procline command 82 is active initially on the anterior teeth 13-23. The Procline command 82 comes to an end on the anterior targeted teeth 180 and the Retrude command 82 is applied to the anterior targeted teeth 180. The Procline command 82 overlaps in time with the Distalize command 82, and the Distalize command 82 overlaps in time with the Retrude command 82. Lastly, the Distalize command 82 on the right posterior teeth 14-17 may be applied longer than the Distalize command 82 on the left posterior teeth 24-27. However, while there is some overlap in time, the Procline, Distalize, and Retrude do not overlap in targeted teeth 180. As shown, the Procline and Retrude commands are applied to the anterior teeth 13-23, and the Distalize command 82 is applied to the posterior teeth 14-17 and 24-27.

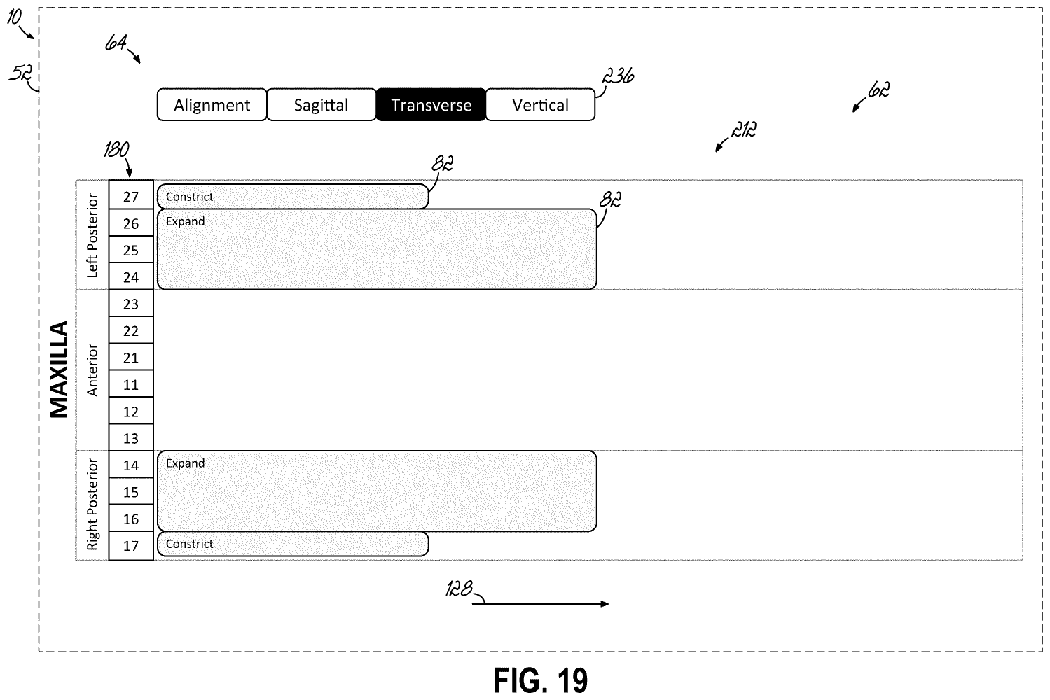

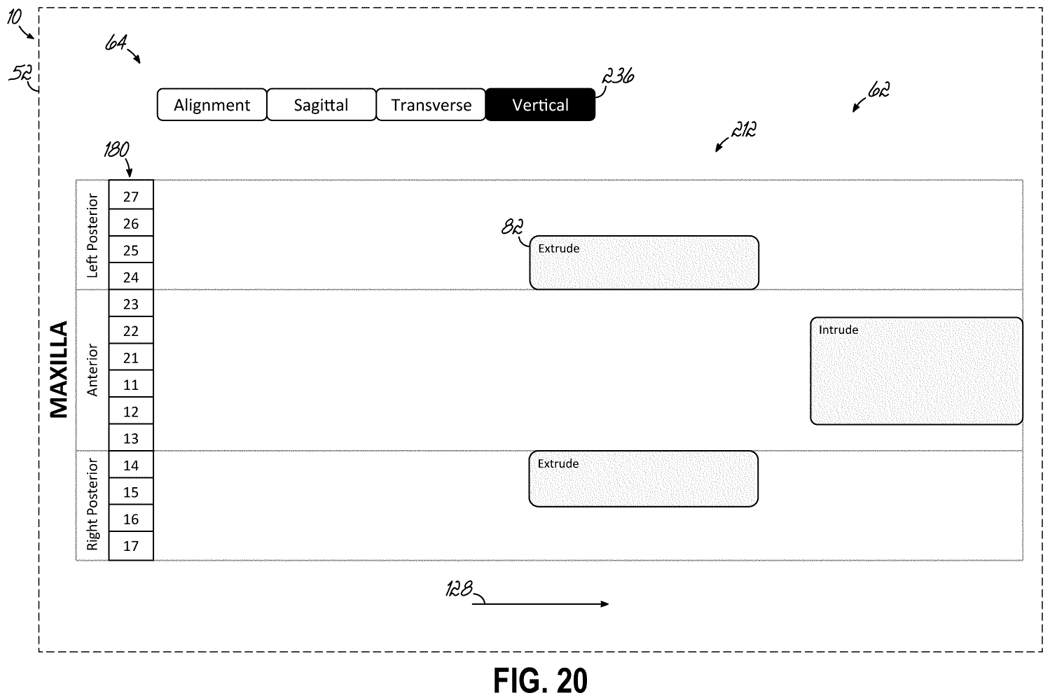

Each of FIGS. 19 and 20 depict different selections on the layer selection bar 236. In FIG. 19, for example, commands categorized under “Transverse” are shown and in FIG. 20, for example, commands categorized under “Vertical” are shown. Other selections on the layer selection bar 236 are possible. For example, any three of Alignment, Sagittal, Transverse, and Vertical are selectable to overlap 3 categories. Further any two of these categories are selectable to overlap 2 categories. In this way, the clinician may evaluate the synchronization of various combinations of the commands 82, 112 with time period 128 and with the targeted teeth 180.

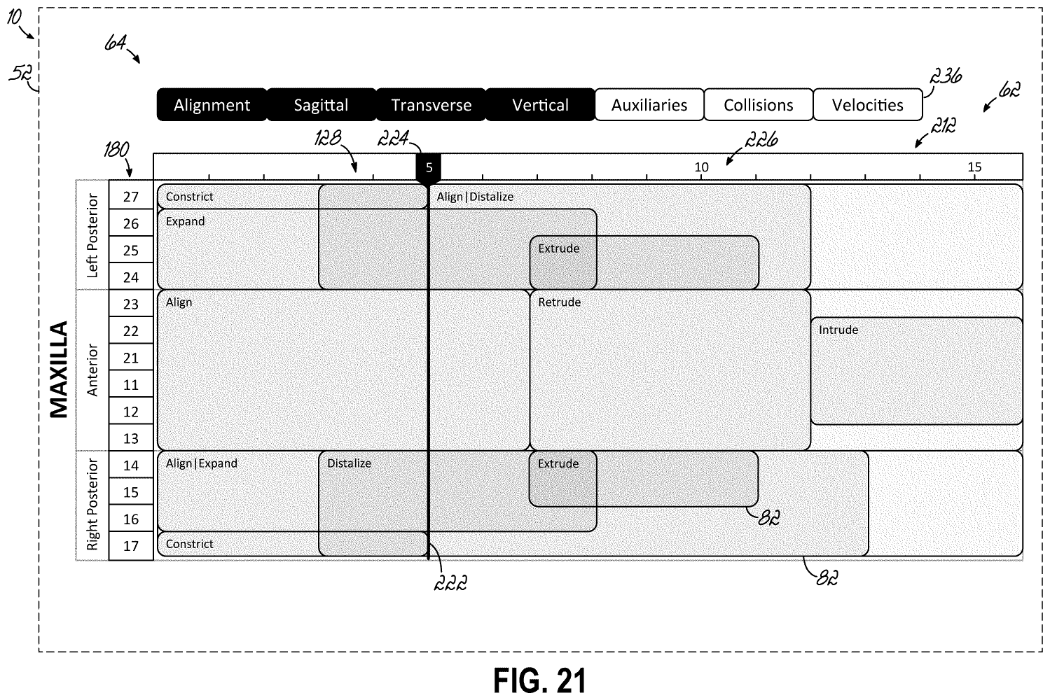

The layer selection bar 236 is not limited to the categories shown in FIGS. 16-20. For example, and with reference to FIG. 21, additional categories are shown on the layer selection bar 236. In FIG. 21, there are 7 layers available for selection in any combination including additional categories “Auxiliaries,” “Collisions,” and “Velocities.” Any of the integrated staging charts 212 shown in FIGS. 16-21 may appear in the user interface 64 described herein, for example those shown in FIGS. 13-15. In addition, the integrated staging chart 212 of FIG. 21 displays additional information similar to that shown in FIG. 15 in which the plurality of stages 226 is shown and in which a single active stage 224 is shown by slider 222. With this information, the clinician may more easily determine commands 82 that are active at any particular stage (i.e., time) and with reference to targeted teeth 180.

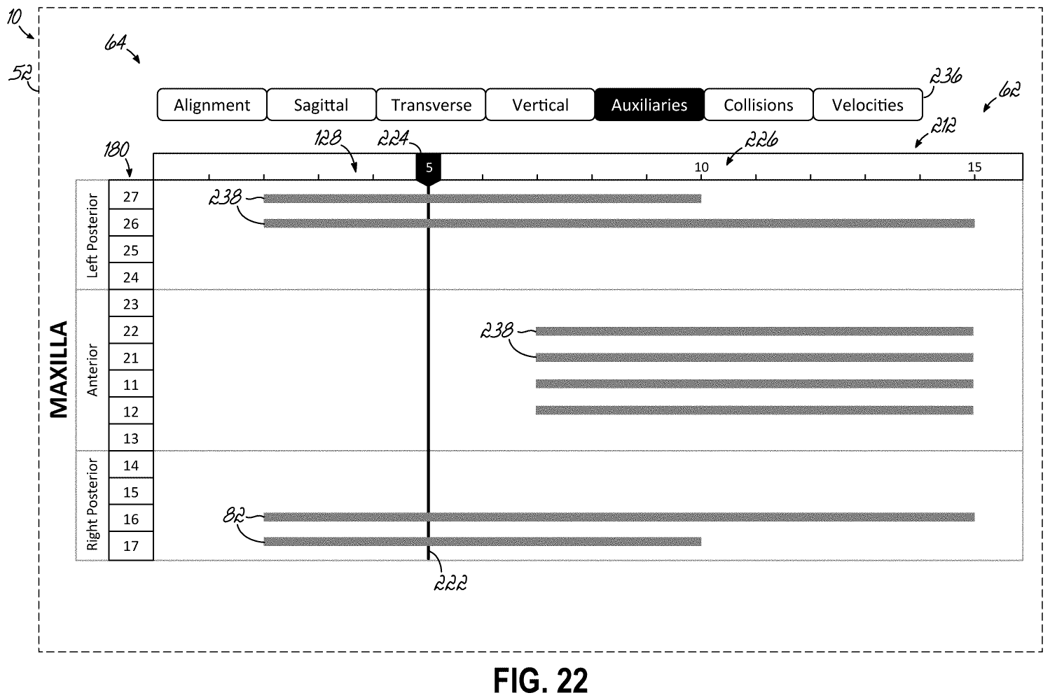

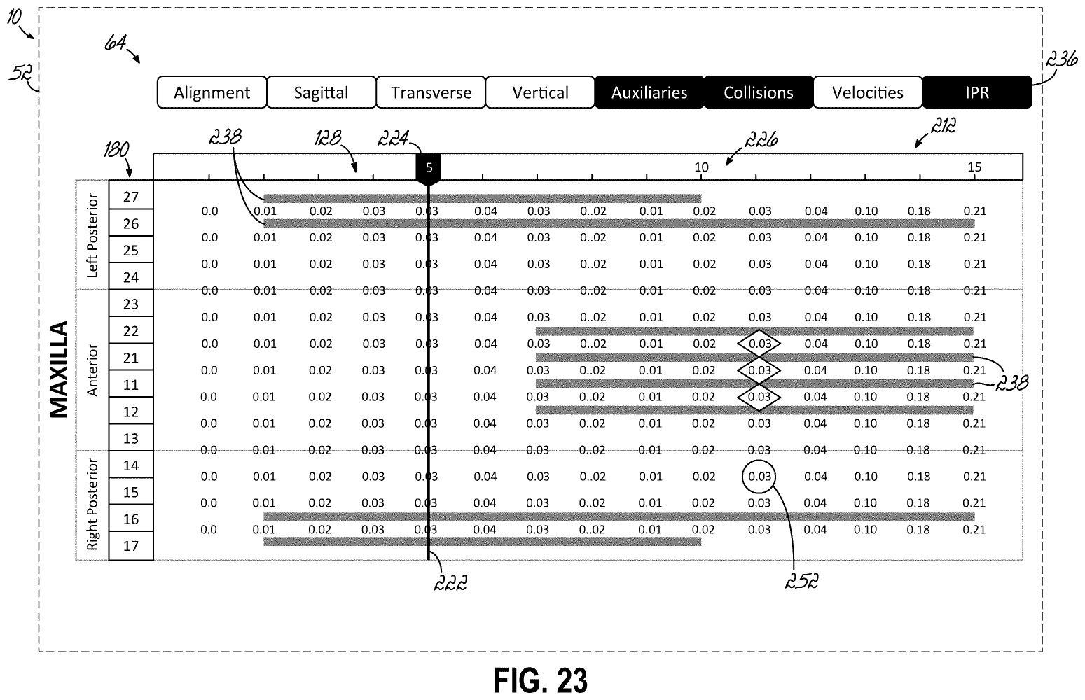

In one embodiment, and with reference to FIGS. 22 and 23, selection of Auxiliaries from the layer selection bar 236 may provide additional information. In the exemplary embodiment of FIG. 22, selection of Auxiliaries in the layer selection bar 236 provides bars 238 or other indica in the integrated staging chart 212 showing the targeted teeth 180 and the time during which auxiliaries, such as attachments 240 in FIG. 15, are prescribed. In the exemplary embodiment shown in FIG. 23, in addition to the bars 238, adjacent between-teeth spacing 252 (e.g., “0.02”) is also overlaid on the integrated staging chart 212.

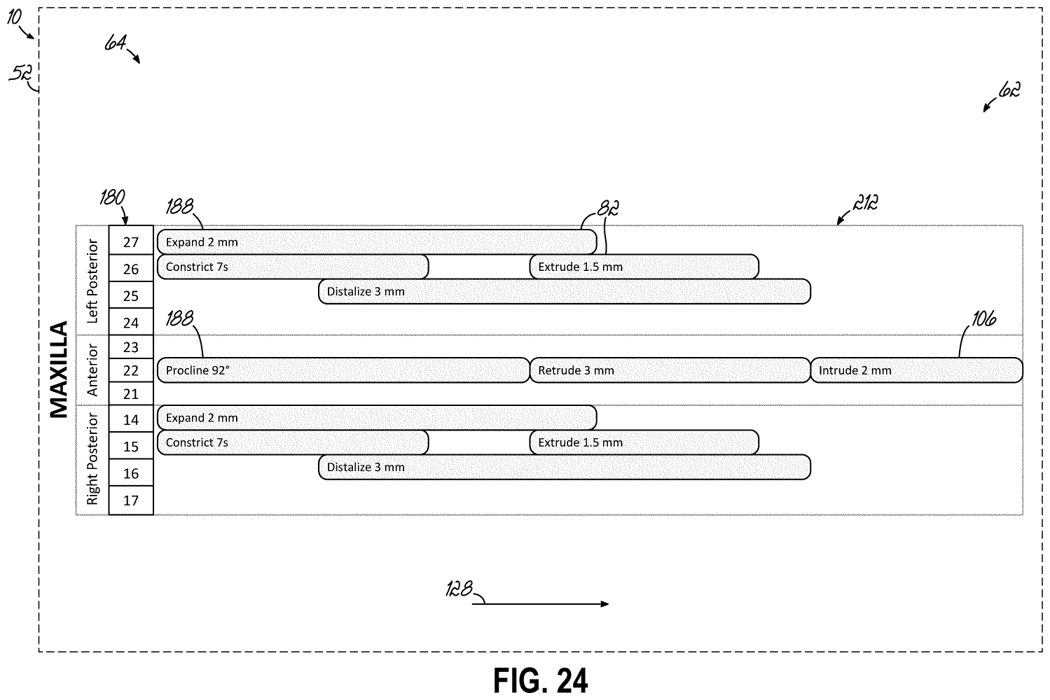

An exemplary embodiment of the integrated staging chart 212 is also shown in FIG. 24 in which the commands 82, 112 are shown with additional information in each block 106. For example, the Expand command 82 is shown depicted with the selected movement parameter 188 of 2 mm. FIG. 24 depicts a “condensed” view. Each command 82 is depicted in a thinned line and only with essential information displayed. The chart 212 shown is designed to avoid taking up too much visual space on the display 52.

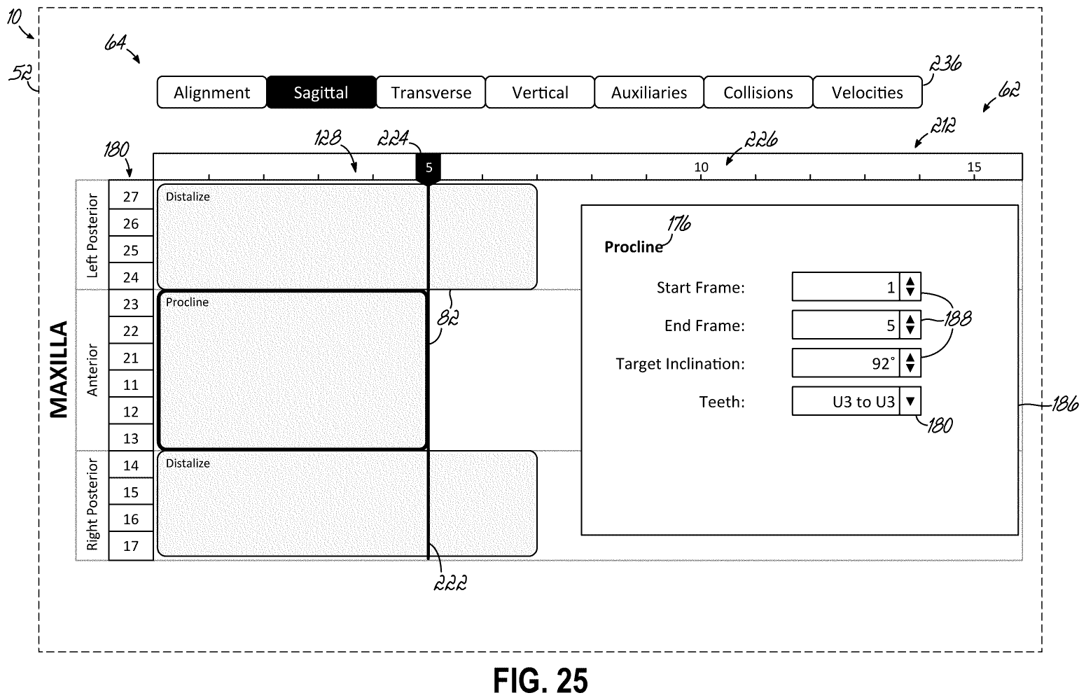

In one embodiment, in FIG. 25, selection of individual ones of the commands 82, 112 may open the details window 186 similar to that shown in FIG. 15 on which the clinician may adjust one or more movement parameters 188. For example, selection of the Procline command 82 may open the details window 186 for the Procline command 82. Each of the movement parameters 188 for the Procline command 82 for Start Frame (a time period), End Frame (a time period), Target inclination, and targeted teeth 180 are modifiable in the details window 186. As shown, the slider 222 is shown at stage 5.

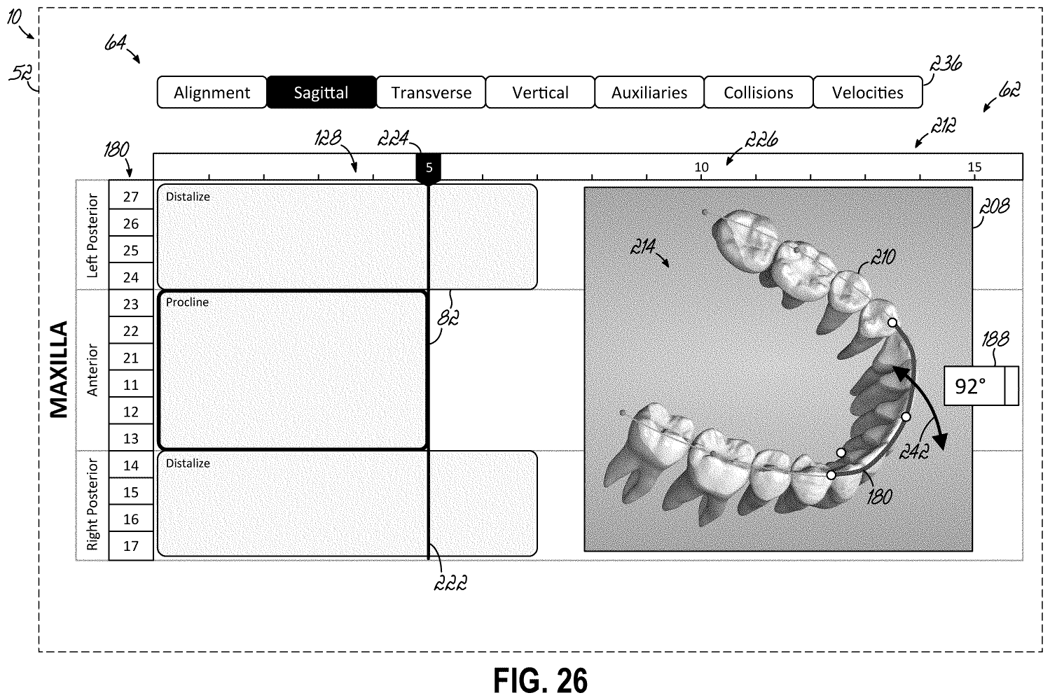

Alternatively, or in addition to the details window 186 shown in FIG. 25, selection of any single one of commands 82 on the integrated staging chart 212 may open the sample movie window 208 as shown in FIG. 26. Application of the selected command 82 may be shown by way of a movie with the movement parameters 188 display proximate the targeted teeth 180. Schematic movement according to that parameter may be shown by way of double arrow 242. In FIG. 26, the targeted teeth 180 and movement parameters 188 are adjustable so that the changes to the movement parameters 188 are visually displayed relative to the targeted teeth 180.

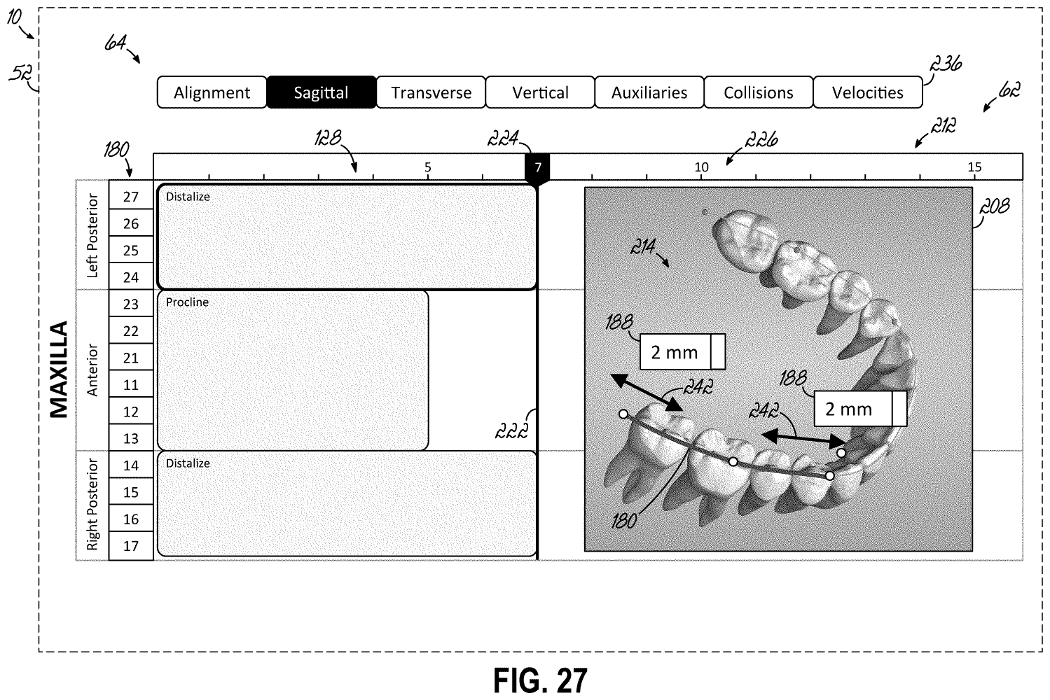

As another example, and with reference to FIG. 27, selection of the left posterior Distalize command 82 on the integrated staging chart 212 may open the sample approver movie window 208. Revisions to the movement parameters 188 for the Distalize command 82 may be shown by way of a movie or tooth movement in the teeth 210 in window 208. In FIG. 27, the targeted teeth 180 and movement parameters 188 are numerically adjustable in the window 208. The clinician is able to revise the targeted teeth 180 and movement parameters 188 and view modifications in near real time in the teeth 210. Movement in FIG. 27 is schematically shown by double arrow 242.

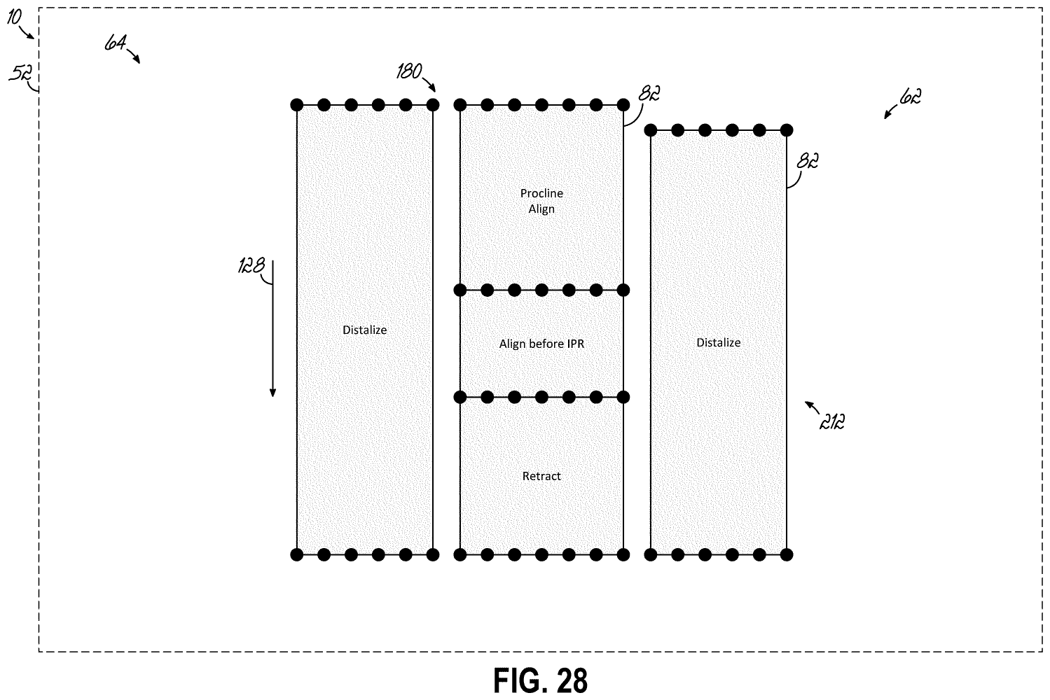

With reference now to FIG. 28, an exemplary embodiment of the integrated staging chart 212 is shown. Display of the integrated staging chart 212 may include a vertical time period 128 and may be by preference of the clinician. In that regard, the vertical orientation of the time period 128 of the prescription 62 is a reorientation of the time period 128 shown horizontally in prior figures. Time is therefore shown in FIG. 28 to increase from top to bottom. Embodiments of the invention are not limited to top to bottom increase in time. Time may be displayed in a manner in which time increases from bottom to top of FIG. 28. With time increasing from top to bottom in FIG. 28, the prescription 62 begins application of the right Distalize command 82 after beginning application of the left Distalize command 82. Application of the Procline command 82 in the center overlaps the Align command 82 but each command 82 is applied beginning at the same time as the left Distalize command 82.

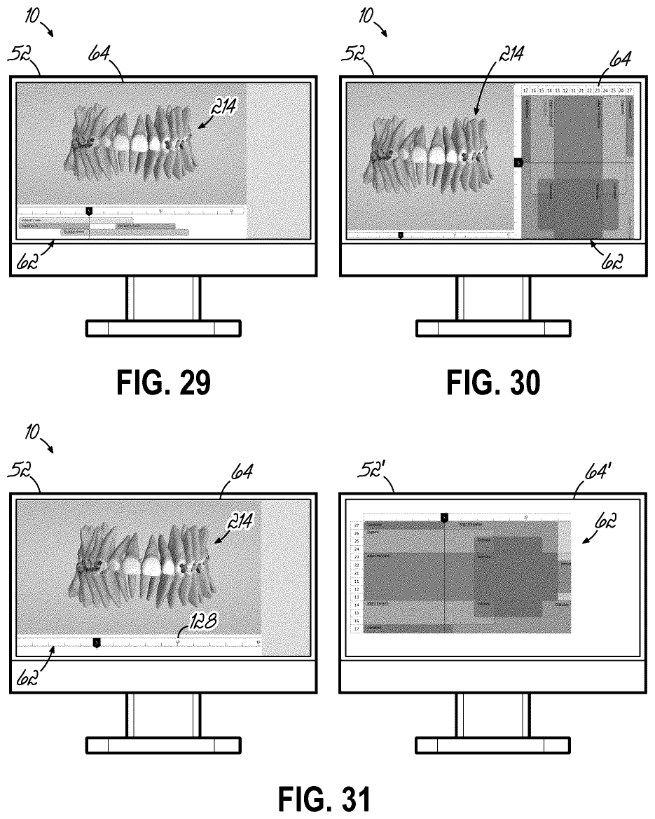

Further in regard to orientation, and with reference to FIGS. 29 and 30, the clinician may opt to display the user interface 64 with the prescription 62 shown horizontally (FIG. 29) or vertically (FIG. 30) on more than one display 52, such as dual displays 52 and 52′ in FIG. 31. Embodiments of the orthodontic treatment planning and manufacturing system 10 are not limited to the number of displays 52, 52′ or the division of the user interface 64 between any number of displays 52, 52′.

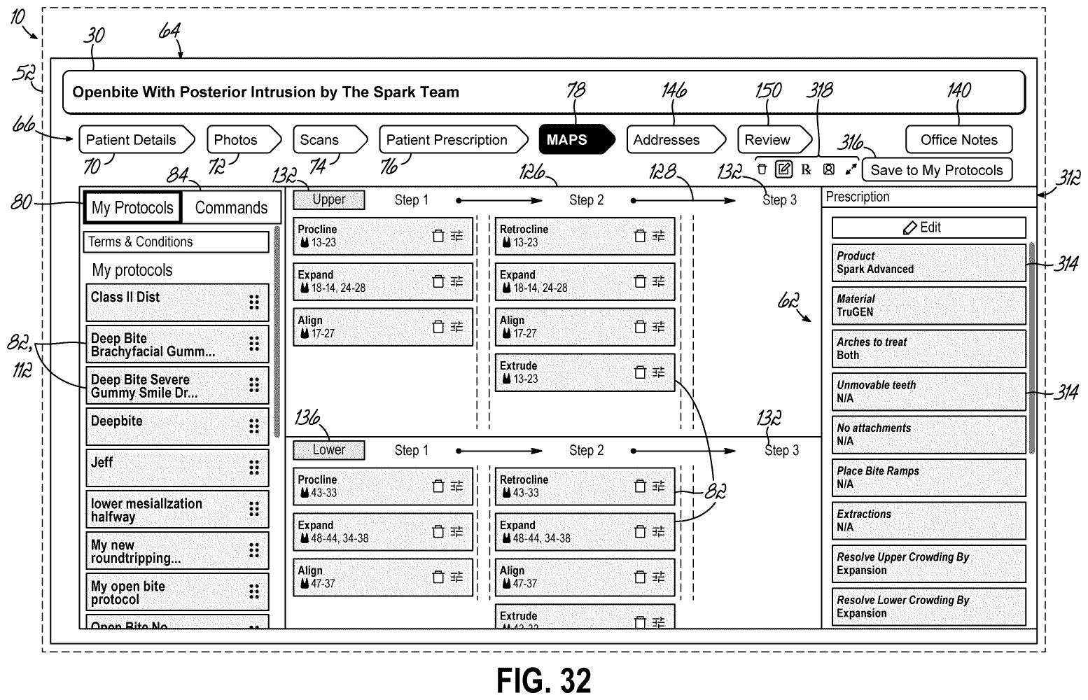

With reference to FIG. 32, in one embodiment, the user interface 64 includes a prescription summary 312 (shown on the right side of the user interface 64). The clinician can view, insert, and remove basic characteristics 314 of the prescription 62 in the prescription summary 312. As shown, the basic characteristics 314 of the prescription 62 include one or more of product information, material type, arches to be treated, and other information usable by the orthodontist and/or by the manufacturer of the orthodontic appliance. Further, the user interface 64 may include a save selection 316 by which the orthodontist may save any of the commands 82, 112 and/or arrangement of commands 82, 112 to the orthodontist's My Protocols block 80. The user interface 64 further includes addition icons 318 by which the user can select alternative windows. For example, the icons 318 are selectable to delete the prescription 62 (shown as a trashcan icon), add notes to the prescription 62 (shown as a notepad icon), display a prescription form (shown as the Rx icon), and to display patient photos (shown as a profile photo icon).

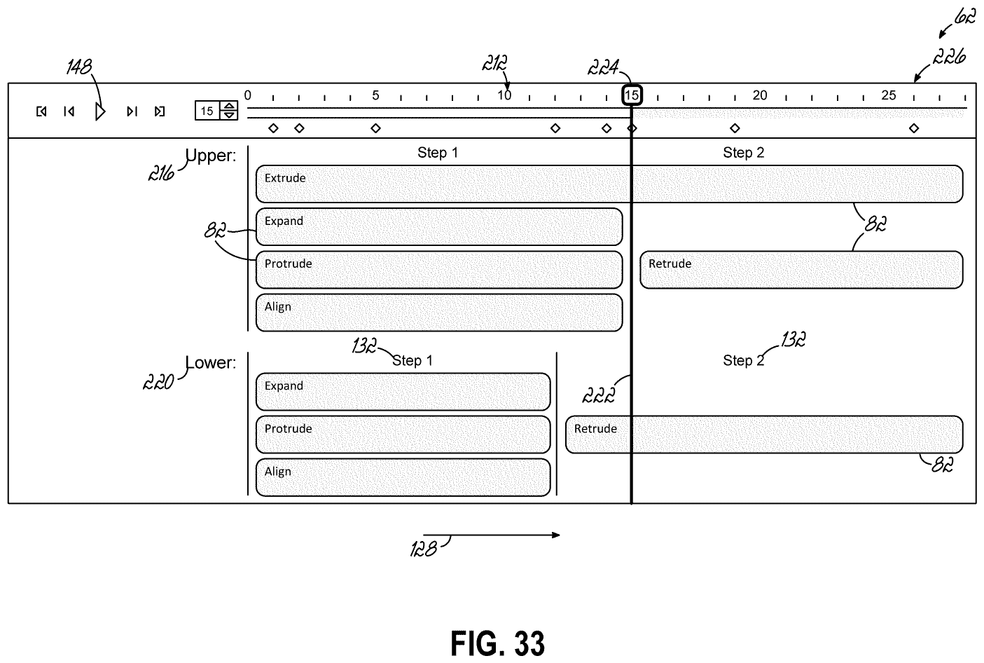

With reference to FIGS. 33, 34, 35, 36, 37, 38, 39, 40, and 41, exemplary embodiments of the user interface 64 include the staging chart 212 integrated with the steps 132 of the prescription 62. FIG. 33 illustrates that the steps 132 may cover multiple stages 224 of the plurality of stages 226, and steps 132 in the upper arch chart 216 do not necessarily correspond to the steps 132 in the lower arch chart 220. In the example shown, Step 1 in the upper arch chart 216 spans from stage zero to stage 15. While FIG. 33 depicts a visual gap between successive commands, e.g., space between the Protrude command 82 and the Retrude command 82 in each of the upper arch chart 216 and the lower chart 220, the gap is for visual appeal and aids in visual manipulation of the commands. For example, the gap facilitates a drag-and-drop type of visual placement of another command between any two successive commands. In the lower arch chart 220, Step 1 spans from stage 0 to stage 12. Similarly, Step 2 in each of the upper arch chart 216 and lower arch chart 220 do not span the same number of stages. In this example, Step 2 in the lower arch chart 220 spans from stage 12 to stage 28 or a total of 16 stages, whereas Step 2 of the upper arch chart 216 spans from stage 15 to stage 28 or a total of 13 stages.

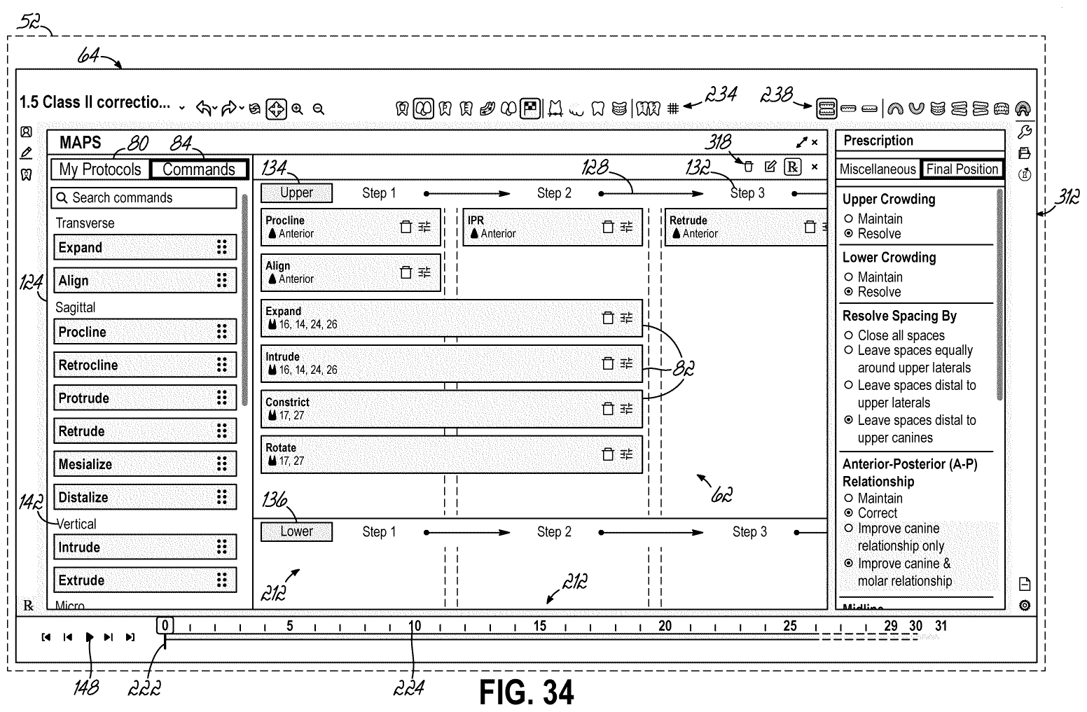

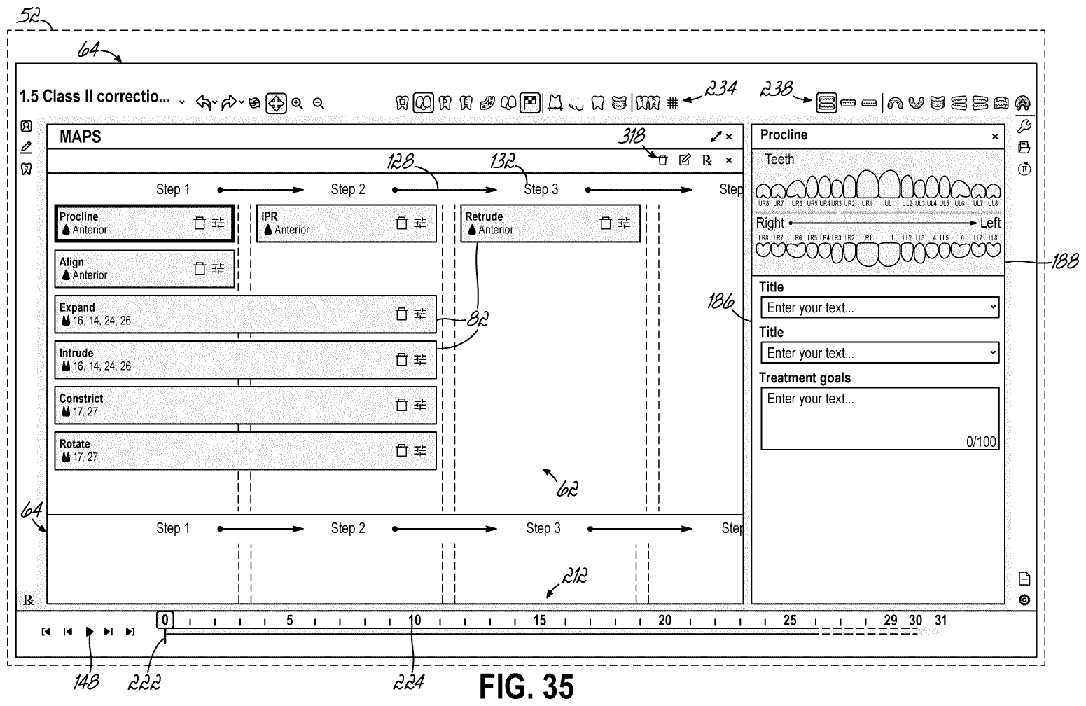

Rather than an overlapping staging chart 212 and steps 132 as shown in FIG. 33, exemplary embodiments of the user interface 64 display the staging chart 212 positioned apart from the steps 132 and commands 82, 112 of the prescription 62 with additional information. As an example, with reference to FIG. 34, the sidebar menu 124 with each of the My Protocols block 80 and commands library 84 and the prescription summary 312 is shown because the Rx icon of the additional icons 318 is active. The staging chart 212 is shown at the bottom of the user interface 64. Tools 234 and 238 are also shown for opening the model 214. A similar embodiment of the user interface 64 is shown in FIG. 35. The details window 186 is shown without the sidebar window 124 shown in FIG. 34.

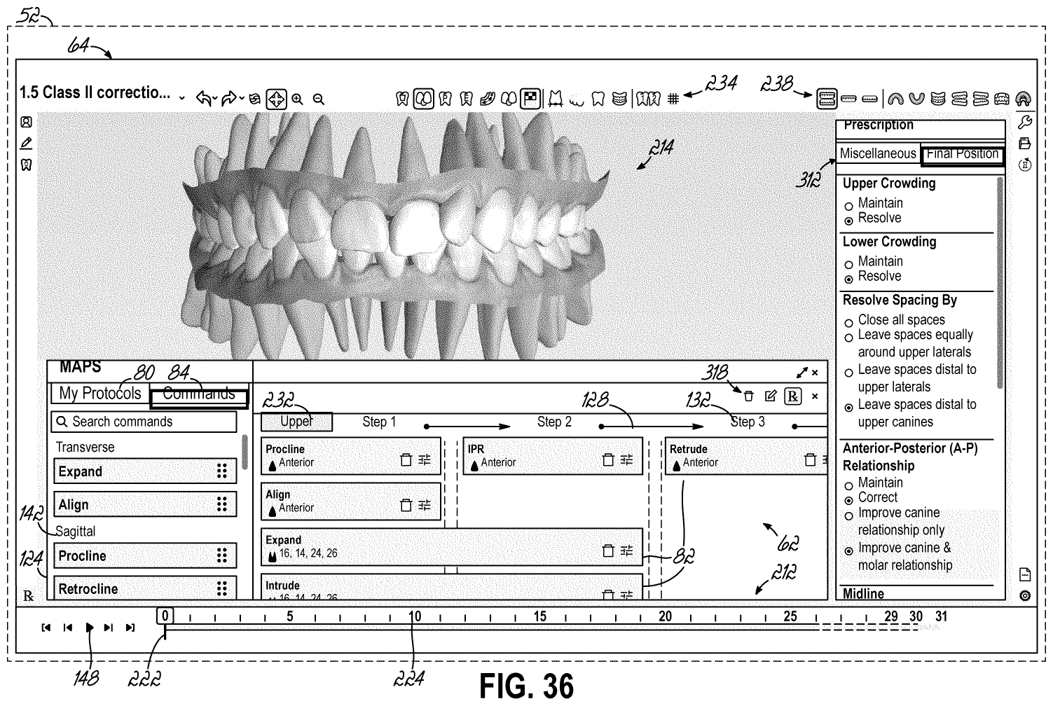

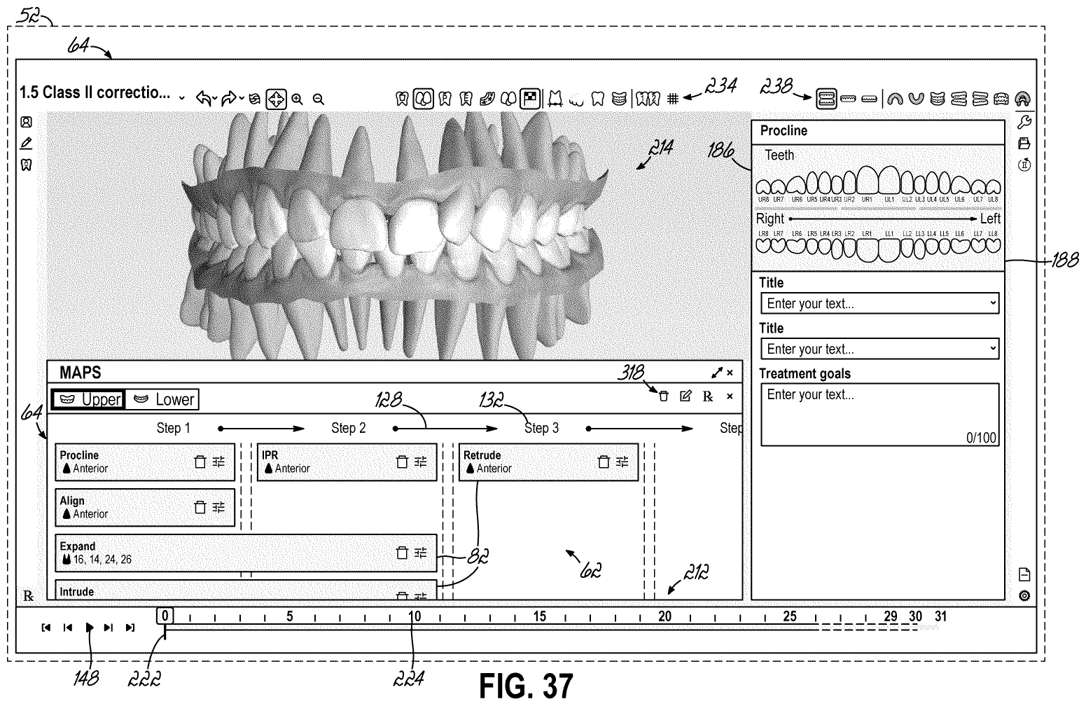

In FIG. 36, the virtual model 214 is shown together with the sidebar window 124, depicting the protocol block 80 and command library 84, and the commands 82 arranged in steps 132 according to the prescription 62. With the Rx icon of the additional icons 318 active, the prescription summary 312 is shown. The staging chart 212 is also shown at the bottom of the interface 64. A similar interface is shown in the example of FIG. 37 in which the details window 186 is displayed in place of the prescription summary 312. The commands 82 are arranged in steps 132 according to the prescription 62 for the upper arch 232. And, the staging chart 212 is shown at the bottom of the user interface 64.

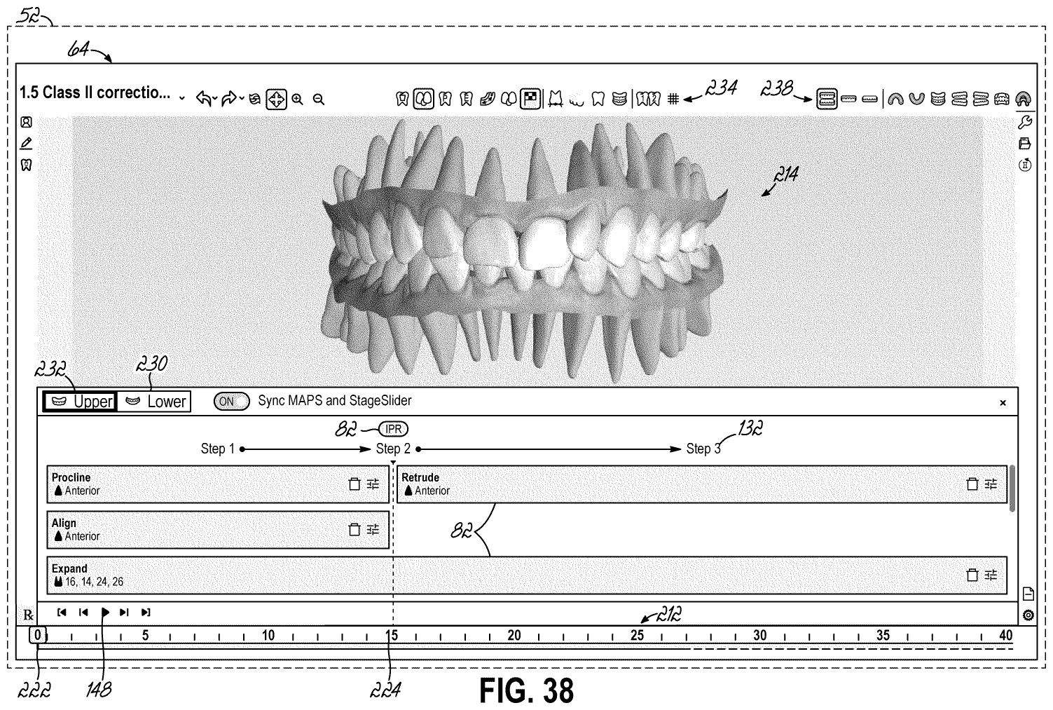

In another similar example, and with reference to FIG. 37, the virtual model 214 is shown together with the details window 186. The commands 82 are arranged in steps 132 according to the prescription 62. The staging chart 212 is shown at the bottom of the user interface 64. In yet another example, and with reference to FIG. 38, the virtual model 214 is shown together with the commands 82 arranged in steps 132 for the upper arch 232. The staging chart 212 is shown at the bottom of the user interface 64.

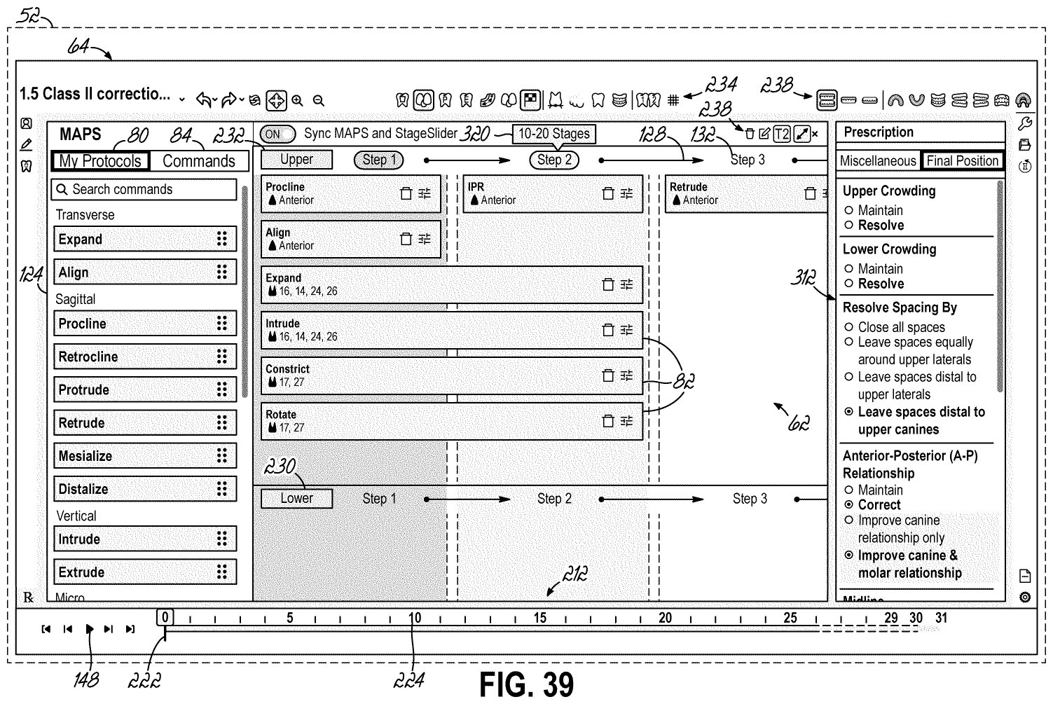

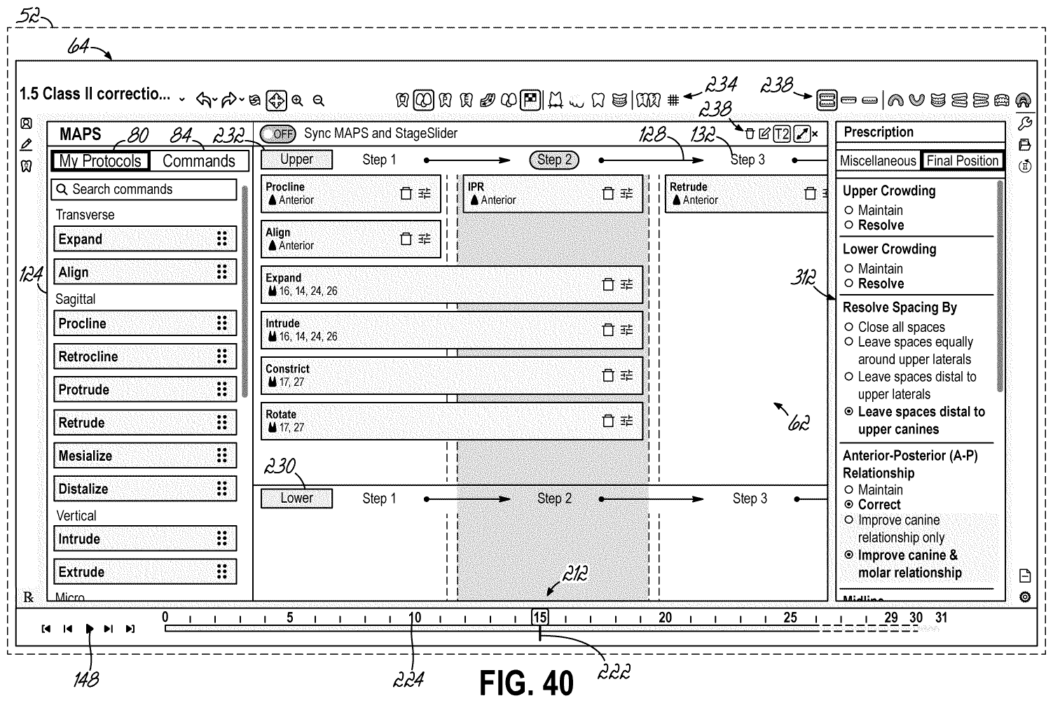

In the exemplary embodiment shown in FIGS. 39 and 40, the user interface 64 includes the sidebar menu 124 and the commands 82 arranged in steps 132 according to the prescription 62. The prescription summary 312 is displayed as is the staging chart 212. In the embodiment shown, hovering the pointer of a “step” depicts at 320 the related stages 224 shown in the staging chart 212.

In the exemplary embodiment shown in FIG. 41, the steps 132 are replaced by stages 224 corresponding to the staging chart 212. As shown, for the upper arch 232, the commands 82 are overlaid with the stages 224 according to the prescription 62. While various embodiments of the invention are shown with respect to placement windows relative to the virtual model and staging chart, embodiments of the user interface 64 are not limited to the arrangement shown.

Referring now to FIG. 1, in one embodiment, after submission of the prescription 62 and treatment protocol, the technician 26 may review the data records 34, including the prescription 62 and treatment protocol and imagery information 24. This data may be received into the input computer 244 specifically dedicated to the design of the appliance 60. The technician 26 may also add input to or control operation of appliance manufacturing equipment 22 controlled by computer 244, such as machine controller 246 and/or to manufacture the appliance 60. Where the inputting, design, and manufacture are performed in the manufacturing system 14, the computers 244, 246, and 248 may be the same computer or separate computers or controllers that are linked to each other or otherwise exchange data 250.

The imagery information 24 received from imaging system 16 may be reviewed by the technician 26 and entered into the input computer 244. The technician 26 via computer 244 may manipulate the imagery information 24 to provide the T1 model 54 and the T2 model 56. The technician 26 may also construct a treatment plan. The treatment plan may include a staging chart that is the integrated staging chart 212 or a direct adaptation of the integrated staging chart 212 and may be a precise prediction of the prescribed treatment based on the T1 model 54 and the T2 model 56 in conjunction with the data records 34, including the prescription 62, from the orthodontist 18. The proposed treatment, the T1 model 54, the T2 model 56, and the treatment plan are communicated to the orthodontist 18 through a network 162 (FIG. 1A) between manufacturing system 14 and the office 12. The orthodontist 18 may modify the initial treatment plan in response to which the design computer 248 recalculates the final treatment positions of the teeth and generates display data for further review, revision, or approval by the orthodontist 18. Algorithms may also be used to determine the treatment plan from the commands 82, 112 of the prescription 62 together with the treatment protocol with little or no technician interaction. That is, the prescription 62 in the form of commands 82 arranged and sequenced per the treatment protocol may be machine-readable code and so dispense with the need for a technician. Advantageously, this improves the efficiency of the orthodontic treatment planning and manufacturing system 10. Alternatively, the prescription 62 and treatment protocol may be converted to machine-readable code by the technician. In this case, the input computer 244 may automatically construct a treatment plan based on at least the prescription 62 and the T1 model 54. That information may be automatically transferred to the orthodontist for approval review.

Once the tooth treatment positions are approved by the orthodontist 18, the computer 156 automatically designs one or more appliances 60, 172 or molds for manufacturing appliances under the supervision of the technician 26. As a digital design is produced, the design information, which includes three-dimensional design display and numerical design data, may be provided over the network 162 to the computer 38 for interactive adjustment and ultimately approval by the orthodontist 18.

When the design has been approved by the orthodontist 18, the analysis and design computer 248 may produce archive files 164 that are written with all the relevant information of the analysis and the history and prescribed treatment of the patient 30. Calculated information for the patient 30 may be stored in a patient data file. From the calculations, the manufacturing computer 156 produces machine-readable code 166 for operating digitally controlled manufacturing equipment 22 to produce the exemplary appliance(s) 60, 172. The machine-readable code 166 may be based on the prescription 62 and treatment protocol and, as such, may include all or any single one of the commands 82, 112 found in the prescription 62 as well as timing associated with the time period 128. For example, the orthodontist may build the prescription 62 with a Use TruGen XR command 82 in which case the machine-readable code 166 based on the prescription 62 may incorporate the TruGen XR command verbatim from the prescription 62.

For manufacture of orthodontic appliances, the manufacturing equipment 22 preferably includes forming machinery 170 which produces the appliances, such as orthodontic brackets 172 themselves, or molds for the appliance 60. Automated bracket or mold making can be carried out by casting or molding of the brackets from molds made by the automated machines, by cutting slots at calculated angles or machining other features in preformed blanks, such as with CNC machinery 174, or by other automated bracket making methods. The forming machinery 170 may shape the surfaces of preformed bracket bases, providing a design option of torquing the teeth by either the bracket slot or base, as may be best for various bracket materials. The forming machinery 170 may also include an appliance archwire bending machine or other type of wire forming machine to produce custom shaped archwires for the appliance 172.

With reference to FIG. 42, as a result, the aligner 60 includes a hollow shell 300 that is configured to encapsulate one or more crowns of a patient's teeth. The shell 300 is formed with a plurality of cavities 302 that collectively define an edge 304, which defines an opening 306. Each cavity 302 is shaped to receive a specific one of the patient's teeth through the opening 306 during use of the aligner 60. The shell 300 is made of an elastic material in one or more layers and may include one or more receptacles 308 that are configured to receive an attachment (not shown) on the patient's tooth and/or one or more devices in the aligner 60. In that regard, the library 84 may include a command 82 to add an attachment to a specified tooth in the prescription 62. The appliance(s) are shipped to the orthodontist 18 or the patient 30 for orthodontic treatment.

During orthodontic treatment, the aligner 60 is selectively positioned over the patient's teeth and may fit tightly due to slight differences in the position of one or more of the cavities 302 relative to the corresponding tooth. A forcible contact with the aligner 60 may move the patient's teeth toward a predetermined position according to a patient's treatment plan that may ultimately end at T2. A set of aligners (not shown) may include one or more aligners 60. During orthodontic treatment, each stage of treatment may include an aligner that progressively moves one or more of the patient's teeth incrementally toward a desired final arrangement. The individual aligners are utilized in a predetermined sequence according to the treatment plan approved by the orthodontist 18 to complete orthodontic treatment or move the patient's teeth to T2. Accordingly, each aligner in the series may move one or more teeth a prescribed amount. While similar, each aligner is slightly different in shape. Cumulatively, these individual amounts may result in complete treatment of the patient's malocclusion.

In general, the routines and instructions executed to implement the embodiments of the invention, whether implemented as part of an operating system or a specific application, component, program, object, module or sequence of instructions, or a subset thereof, may be referred to herein as “computer program code,” or simply “program code.” Program code typically comprises computer-readable instructions that are resident at various times in various memory and storage devices in a computer, such as, any one of or a combination of computers 38, 244, 246, 248 or in the appliance manufacturing equipment 22 and that, when read and executed by one or more processors in a computer, cause that computer to perform the operations necessary to execute operations and/or elements embodying the various aspects of the embodiments of the invention. Computer-readable program instructions for carrying out operations of the embodiments of the invention, such as the arrangement of elements in the interface 64 and display of interface 64 on display 52 may be, for example, assembly language or either source code or object code written in any combination of one or more programming languages.

Various program code described herein may be identified based upon the application within which it is implemented in specific embodiments of the invention. However, it should be appreciated that any particular program nomenclature which follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), it should be appreciated that the embodiments of the invention are not limited to the specific organization and allocation of program functionality described herein.

The program code embodied in any of the applications/modules described herein, such as, the prescription 62 of commands 82, 112 or the commands 82, 112 themselves and the treatment protocol is capable of being individually or collectively distributed as a program product in a variety of different forms. In particular, the program code may be distributed using available means for distribution, including direct download from an internet accessible computer or via a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of data, such as computer-readable instructions, data structures (e.g., imagery information 24, 3-D digital model 54 and 56, prescription 62 and treatment protocol, user interfaces 52, protocol block 80, and library 84), program modules, or other data. Computer-readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired data and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an orthodontic appliance including instructions that implement the functions, acts, and/or operations specified in flow-charts, sequence diagram, and/or block diagrams. The computer program instructions may be provided to one or more processors of a general-purpose computer, a special-purpose computer, or other programmable data processing apparatus to produce a prescription and/or an appliance, such that the instructions, which execute via the one or more processors, cause a series of computations to be performed to implement the functions, acts, and/or operations specified in the flow-charts, sequence diagrams, and/or block diagrams.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

1. A system for orthodontic treatment planning for a patient, the system comprising:
a processor;
memory coupled to the processor, the memory configured to store computer-program instructions that, when executed by the processor, cause the system to:
display a user interface on a display, the user interface being for a user to input a prescription for treatment of the patient, the user interface displaying a plurality of commands for selection, wherein at least one command of the plurality of commands is a predetermined instruction based on orthodontic nomenclature for moving or modifying one or more of a patient's teeth; and
receive a first command of the at least one command of the plurality of commands into the prescription for treatment,
wherein receipt of the first command into the prescription for treatment defines a treatment protocol for application of the prescription for treatment to the patient's teeth, and
wherein the treatment protocol includes a first time period during which the first command is applied to the patient's teeth, the first time period being during a treatment period over which the prescription for treatment is to be applied to the patient's teeth.

2. The system of claim 1, wherein the memory is configured to store computer-program instructions that, when executed by the processor, cause the system to:
after receipt of the first command, receive a second command of the at least one command of the plurality of commands into the prescription for treatment,
wherein receipt of the second command into the prescription of treatment adds a second time period to the treatment protocol,
wherein the second time period determines when the second command is applied to the patient's teeth, and
wherein the first time period is before or is simultaneous with the second time period.

3. The system of claim 2, wherein the treatment protocol is defined by a plurality of steps including a first step and a second step,
wherein receipt of the first command sets the first time period to be equal to the first step and the second step, and
wherein receipt of the second command sets the second time period to be equal to the first step whereby the first command and the second command are applied to the patient's teeth simultaneously during the first step.

4. The system of claim 3, wherein the second time period does not include the second step whereby the first command is applied to the patient's teeth in the second step and the second command is not applied to the patient's teeth in the second step.

5. The system of claim 2, wherein when executed by the processor, the computer-program instructions cause the system to:
generate a plurality of stages so that one or both the first time period and the second time period are defined by one or more of the plurality of stages.

6. The system of claim 5, wherein when executed by the processor, the computer-program instructions cause the system to:
display, in the user interface, an integrated staging chart, wherein the integrated staging chart depicts at least the first command and depicts the first time period to be equal to one or more of the plurality of stages,
wherein the integrated staging chart defines at least a portion of the orthodontic treatment plan.

7. The system of claim 6, wherein the integrated staging chart displays the first time period and the second time period sequentially with the first command being shown as being applied to the patient's teeth before the second command.

8. The system of claim 7, wherein when executed by the processor, the computer-program instructions cause the system to:
display, in the user interface, the plurality of steps overlaid with the plurality of stages.

9. The system of claim 5, wherein each stage of the plurality of stages corresponds to planned orthodontic treatment with an appliance.

10. The system of claim 1, wherein the treatment protocol is defined by a plurality of steps, and
wherein receipt of the first command into the prescription for treatment sets the first time period to be at least one step of the plurality of steps.

11. The system of claim 1, wherein the treatment protocol is defined by a plurality of steps, and
wherein receipt of the first command into the prescription for treatment sets the first time period to be equal to all steps of the plurality of steps.

12. The system of claim 1, wherein when executed by the processor, the computer-program instructions cause the system to:
display, on the user interface, a message that a displayed command of the plurality of commands is locked against being received in the prescription for treatment.

13. The system of claim 1, wherein when executed by the processor, the computer-program instructions cause the system to:
display, on the user interface, a virtual model of teeth and controls operable by the user to cause the processor to simulate application of tooth movement according to at least one of the first command and the second command to the virtual model of teeth.

14. The system of claim 1, wherein when executed by the processor, the computer-program instructions cause the system to:
display, in the user interface, a virtual model of patient's teeth and one or more controls operable by the user to cause the processor to simulate application of the prescription for orthodontic treatment of the patient to the virtual model of the patient's teeth.

15. The system of claim 1, wherein the first command includes one or more tooth movement parameters, when executed by the processor, the computer-program instructions cause the system to:
display, in the user interface, one or more controls linked to one or more teeth in the virtual model, the one or more controls being operable by the user to cause the processor to modify tooth movement parameters in the first command and/or in the second command.

16. The system of claim 1, when executed by the processor, the computer-program instructions cause the system to:
display, in the user interface, the at least one command of the plurality of commands in a respective rectangular-shaped border encircling only the predetermined instruction and,
wherein each rectangular-shaped border of the respective rectangular-shaped borders encircling the predetermined instruction overlap to form two or more layers of commands.

17. A computer-implemented method of creating an orthodontic treatment plan applicable to teeth of a patient, the method comprising:
receiving a digital model of a patient's teeth in a first arrangement;
displaying a plurality of commands from a library of commands, wherein the library of commands is predetermined and at least one command of the displayed plurality of commands is based on orthodontic nomenclature for moving or modifying one or more of the patient's teeth; and
placing a first command of the at least one command into a prescription for treatment,
wherein placing the first command defines a treatment protocol for application of the prescription for treatment to the patient's teeth; and
wherein the treatment protocol includes a first time period that determines when the first command is applied to the patient's teeth during a treatment period over which the prescription for treatment is to be applied to the patient's teeth.

18. The computer-implemented method of claim 17, wherein after placing the first command, the method further comprises:
placing a second command of the at least one command of the plurality of commands into the prescription for treatment,
wherein placing the second command into the prescription of treatment adds a second time period to the treatment protocol,
wherein the second time period determines when the second command is applied to the patient's teeth, and
wherein the first time period is before or is simultaneous with the second time period.

19. The computer-implemented method of claim 17, wherein the treatment protocol is defined by a plurality of steps, and
wherein placing the first command into the prescription for treatment sets the first time period to all steps of the plurality of steps.

20. The computer-implemented method of claim 19, wherein the plurality of steps includes a first step and a second step,
wherein placing the first command sets the first time period to be equal to the first step and the second step, and
wherein placing the second command sets the second time period to be equal to the first step whereby the first command and the second command are applied to the patient's teeth simultaneously during the first step.

21. The computer-implemented method of claim 19, wherein placing the second command sets the second time period to be equal to the first step only whereby the first command is applied to the patient's teeth in the second step and the second command is not applied to the patient's teeth in the second step.

22. The computer-implemented method of claim 19, the method further comprising:
generating a plurality of stages so that one or both the first time period and the second time period are defined by one or more of the plurality of stages.

23. The computer-implemented method of claim 22, the method further comprising:
displaying an integrated staging chart depicting the first command and the second command and the plurality of stages,
wherein the integrated staging chart defines at least a portion of the orthodontic treatment plan.

24. The computer-implemented method of claim 23, wherein displaying the integrated staging chart includes displaying the first time period and the second time period sequentially with the first command being shown as being applied to the patient's teeth before the second command.

25. The computer-implemented method of claim 22, further comprising:
displaying the plurality of steps overlaid with the plurality of stages.

26. The computer-implemented method of claim 22, wherein each stage of the plurality of stages corresponds to planned orthodontic treatment with an appliance.

27. The computer-implemented method of claim 17, wherein the treatment protocol is defined by a plurality of steps, and
wherein placing the first command into the prescription for treatment sets the first time period to at least one step of the plurality of steps.

28. The computer-implemented method of claim 17, wherein placing one of the at least one command into the prescription for treatment is prevented.

29. The computer-implemented method of claim 18, the method further comprising:
displaying a virtual model of teeth and controls operable by the user to operate the virtual model; and
when activated by the controls, simulating application of tooth movement according to at least one of the first command and the second command to the virtual model of teeth.

30. The computer-implemented method of claim 18, further comprising:
displaying a virtual model of patient's teeth, and
simulating application of the prescription for orthodontic treatment of the patient to the virtual model of the patient's teeth.

31. The computer-implemented method of claim 30, wherein the first command and/or the second command include one or more tooth movement parameters, the method further comprising:
displaying one or more controls selectable by the user and configured to move one or more teeth in the virtual model of the patient's teeth;
wherein selecting the one or more controls and moving the one or more teeth with the one or more controls modifies one or more tooth movement parameters in the first command and/or in the second command.

32. The computer-implemented method of claim 17, further comprising:
displaying the at least one command of the plurality of commands in a respective rectangular-shaped border encircling the predetermined instruction.
